(19) United States
(12) Patent Application Publication
(10) Pub. No.: US 2023/0132973 A1
(43) Pub. Date: May 4, 2023

(54) DIHYDRO-SPIRO[INDOLINE-3:1'-iSOQUINOLIN]-2-ONES AS ANTIMALARIAL AGENTS

(71) Applicant: The University of Buea, BUEA (CM)

(72) Inventors: Noella Molisa Efange, BUEA (CM); Lawrence Ayong, BUEA (CM); Maloba Mabanyi Mesembe Lobe, BUEA (CM); Simon Mbua Ngale Efange, BUEA (CM)

(21) Appl. No.: 17/910,695

(22) PCT Filed: Mar. 16, 2021
(86) PCT No.: PCT/IB2021/052184

§ 371 Date: Sep. 9, 2022

Related U.S. Application Data
(60) Provisional application No. 62/990,718, filed on Mar. 17, 2020.

(51) Int. Cl. A61K 31/4747; A61P 33/06; A61K 45/06

(52) U.S. Cl. CPC A61K 31/4747; A61P 33/06; A61K 45/06

(57) ABSTRACT

The present invention is directed to the antimalarial activity of various 3′,4′-dihydro-2′H-spiro[indoline-3:1′-isoquinolin]-2-ones and related compounds. The present invention is also directed to the use of these compounds as antimalarial agents, in the treatment of malaria. In addition, the invention relates to pharmaceutical compositions comprising one or more of these compounds alone or in combination with other therapeutic agents for the treatment and/or radical cure of severe acute malaria. The invention is also directed to the use of these agents alone and in combination with other agents in malaria prophylaxis, for example, by inhibiting and/or reducing the likelihood of a malaria infection.

### RELATED APPLICATIONS

This application claims the benefit of priority of U.S. provisional application Ser. No. 62/990,718, filed Mar. 17, 2020, the entire contents of which application is incorporated by reference herein.

### FIELD OF THE INVENTION

The present invention is directed to the antimalarial activity of various 3′,4′-dihydro-2′H-spiro[indoline-3:1′-isoquinolin]-2-ones and related compounds. The present invention is also directed to the use of these compounds as antimalarial agents, in the treatment of malaria in patients in need. In addition, the invention relates to pharmaceutical compositions comprising one or more of these compounds alone or in combination with other therapeutic agents for the treatment and/or radical cure of severe acute malaria. The invention is also directed to the use of these agents alone and in combination with other agents in malaria prophylaxis, for example, by inhibiting and/or reducing the likelihood of a malaria infection.

### BACKGROUND OF THE INVENTION

Malaria is a devastating disease that causes considerable morbidity and mortality worldwide and constitutes a major public health problem in many countries. According to the WHO World Malaria Report 2018, an estimated 219 million cases of malaria occurred in 2017 worldwide with 435,000 deaths. While the number of malaria cases reflects a 13% decrease from 2010, it is clear that malaria remains a major public health problem, especially in the African region where 91% of the cases are found. Of particular concern in the African region, between 2016 and 2017 malaria prevalence increased by more than 20% in 10 African countries reporting; only 2 countries reported a reduction in prevalence (World Malaria Report 2018). Recent progress in malaria control is threatened by the emergence of resistance to artemisinin combination therapies (ACTs) (reviewed in Andrews & Odom, 2018; Tse et al., 2019). Therefore, there is a continuing need for newer antimalarials with novel mechanisms of action to reinforce the antimalarial drug armamentarium.

### BRIEF DESCRIPTION OF THE INVENTION

This invention is directed to 3′,4′-dihydro-spiro[indoline-3:1′-isoquinolin]-2-one compounds according to the chemical structure I:

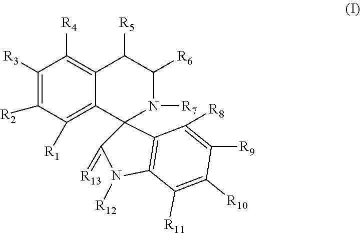

wherein R₁ is H, OH, C₁-C₆ hydroxyalkyl, halo (F, Cl, Br, I), C₁-C₆ alkoxy (often C₁-C₃ alkoxy, more often OMe), (CH₂)ₙCOOH, (CH₂)ₙC(O)C₀-C₆ alkyl, (CH₂)ₙC(O)OC₁-C₆ alkyl, (CH₂)ₙOC(O)C₀-C₆ alkyl or O(CH₂)ₙaryl, aryl, haloaryl, alkoxyaryl (more often phenyl or naphthyl);

R₂ and R₃ are each independently H, OH, C₁-C₆ hydroxyalkyl, halo (F, Cl, Br, I), C₁-C₆ alkoxy (often C₁-C₃ alkoxy, more often OMe), (CH₂)ₙCOOH, (CH₂)ₙC(O)C₀-C₆ alkyl, (CH₂)ₙC(O)OC₁-C₆ alkyl, (CH₂)ₙOC(O)C₀-C₆ alkyl, O—(CH₂)ₙaryl, aryl, haloaryl, alkoxyaryl (more often phenyl or naphthyl), heteroaryl (more often thienyl, furyl, pyrrolyl, pyridyl) or R₂ and R₃ together form a 5- or 6-membered cycloalkyl or heterocyclic group containing 1, 2 or 3 heteroatoms (O, S, or N), preferably, the heterocyclic group formed is a dioxolanyl (3,4-methylenedioxy), dioxanyl (3,4-ethylenedioxy), dithiolanyl, tetrahydrofuranyl, tetrahydrothiophenyl, pyrrolidinyl, imidazolyl, imidazolinyl, imidazolidinyl, tetrahydropyranyl, thienyl, piperidinyl or piperazinyl;

R₄ is H, OH, halo (F, Cl, Br, I), C₁-C₆ alkoxy (often C₁-C₃ alkoxy, more often OMe), (CH₂)ₙCOOH, (CH₂)ₙC(O)C₀-C₆ alkyl, (CH₂)ₙC(O)OC₁-C₆ alkyl, (CH₂)ₙOC(O)C₀-C₆ alkyl, O(CH₂)ₙaryl, (CH₂)ₙAryl (often, phenyl or naphthyl, more often phenyl), (CH₂)ₙHeteroaryl; aryl, alkoxyaryl, haloaryl (more often phenyl or naphthyl), heteroaryl (more often furyl, thienyl, pyrrolyl, pyridyl);

R₅ is H, alkyl (preferably C₁-C₆ alkyl), C₁-C₆ alkoxy, (CH₂)ₙAryl (often, phenyl or naphthyl, more often phenyl), (CH₂)ₙHeteroaryl, aryl (preferably, phenyl, substituted phenyl); heteroaryl (preferably, pyridyl, thienyl, furyl, pyrrolyl);

R₆ is H, alkyl (preferably C₁-C₆ alkyl), trifluoromethylalkyl (preferably C₁-C₅ alkyl), C₁-C₆ alkoxy, (CH₂)ₙAryl (often, phenyl or naphthyl, more often phenyl), (CH₂)ₙHeteroaryl, carboxyl, carbomethoxy, carboxamido, cyano;

R₇ is H, alkyl (preferably C₁-C₆ alkyl), (CH₂)ₙAryl (often, phenyl or naphthyl, more often phenyl), (CH₂)ₙC(O)C₀-C₆ alkyl, —(CH₂)ₙR^{N1}N—C(O)—NR^{N2}R^{N3}, (CH₂)ₙ—S(O)₂Aryl, —OC(O)NR^{N1}R^{N2};

R₈ is H, OH, Halo, Nitro, C₁-C₆ hydroxyalkyl, (CH₂)ₙNR^{N1}R^{N2}, —(CH₂)ₙ—NR^{N1}—(CH₂)ₙ-Aryl (often, phenyl or naphthyl, more often phenyl), —NR^{N1}SO₂Aryl (often, phenyl or naphthyl, more often phenyl), (CH₂)ₙC₃-C₈cycloalkyl-NR^{N1}R^{N2}, C₁-C₆ alkoxy, O(CH₂)ₙaryl, (CH₂)ₙAryl (often, phenyl or naphthyl, more often phenyl), (CH₂)ₙHeteroaryl, C₁-C₆ alkyl, C₂-C₆ vinyl, C₂-C₆ alkynyl, —SO₂NR^{N1}R^{N2}, —OC(O)NR^{N1}R^{N2}, CONR^{N1}R^{N2}, CH₂NR^{N1}R^{N2};

R₉, R₁₀ and R₁₁ are each independently H, OH, Halo, Nitro, C₁-C₆ hydroxyalkyl, piperidyl, pyrrolidyl, morpholinyl, piperazinyl, (CH₂)ₙNR^{N1}R^{N2}, —(CH₂)ₙ—NR^{N1}—(CH₂)ₙ-Aryl (often, phenyl or naphthyl, more often phenyl), —NR^{N1}SO₂Aryl (often, phenyl or naphthyl, more often phenyl), (CH₂)ₙC₃-C₈cycloalkyl-NR^{N1}R^{N2}, C₁-C₆ alkoxy, O(CH₂)ₙaryl, (CH₂)ₙAryl (often, phenyl or naphthyl, more often phenyl), (CH₂)ₙHeteroaryl, C₁-C₆ alkyl, C₂-C₆ vinyl, C₂-C₆ alkynyl, —SO₂NR^{N1}R^{N2}, —OC(O)NR^{N1}R^{N2}, (CH₂)ₙC(O)OC₀-C₆ alkyl or (CH₂)ₙOC(O)C₀-C₆ alkyl, CONR^{N1}R^{N2}, CH₂NR^{N1}R^{N2};

R₁₂ is H, OH, alkyl (C₁-C₆), hydroxyalkyl (preferably C₁-C₆ hydroxyalkyl), an optionally substituted (CH₂)ₙAryl (often, phenyl, benzyl or naphthyl, more often benzyl or naphthyl, the Aryl group being optionally substituted with one or two Halo groups, preferably F, Cl or Br, a nitro, CN or a C₁-C₆, preferably a C₁-C₃ alkyl group, preferably R₁₂ is an optionally substituted benzyl group or naphthyl group), (CH₂)ₙC₃-C₈cycloalkyl, (CH₂)ₙC(O)NR^{N1}Aryl or (CH₂)ₙ—C(O)C₀-C₆ alkyl, aryl (more often phenyl or naphthyl), heteroaryl (more often furyl, thienyl, pyrrolidyl or pyridyl);

R₁₃ is O or S;

R^{N1}, R^{N2} and R^{N3} are each independently H or a C₁-C₆ alkyl group which is optionally substituted with one or two hydroxyl groups and up to three halo groups (preferably F);

n is 0-12, preferably 0-6, often 1-6 or 0, 1, 2 or 3, or

a pharmaceutically acceptable salt, stereoisomer, solvate, polymorph or mixture thereof.

In embodiments, R₁₂ is a phenyl group, a benzyl group or a naphthyl group, each of which is optionally substituted with a C₁-C₆ alkyl group, a nitro group, a cyano group or one or two halo groups (preferably F, Cl or Br).

In embodiments, the compound is a single compound as set forth in FIG. 1 hereof. Often, the compound is a single compound identified as compounds 4a,c,d,f-h,j-l; 5b,d,e-l,m; 6a-c; 7a-d,h,i; 8a-f,h-m,o,p; and 9a-c,h,i; 10; 11 and 12 of FIG. 1.

In embodiments, R₁, R₂ and R₃ are each independently H, halo (preferably F, Cl or Br), methoxy, heterocycloalkyl (preferably piperidyl, pyrrolidyl, morpholinyl or piperazinyl) and R₄ is hydrogen, methyl or phenyl, wherein the methyl group or phenyl group (preferably the phenyl group) is optionally substituted with 1 or 2 halo groups (preferably F, Cl or Br), a nitro group, a CN group or a C₁-C₆ alkyl group, preferably a C₁-C₃ group, most often a methyl group.

In embodiments, the invention is directed to a compound as set forth in FIG. 17 hereof or a pharmaceutically acceptable salt thereof.

In embodiments, the invention is directed to a compound according to chemical structure II:

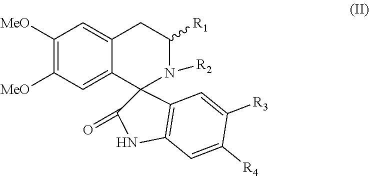

Wherein R₁ is hydrogen or methyl;

R₂ is hydrogen or methyl;

R₃ is halogen, preferably fluorine or chlorine; and

R₄ is halogen, preferably fluorine or chlorine or a nitrogen-containing heterocyle, preferably a 5- to 6-membered heterocycle such as pyrrolidyl, pyrrolyl, piperidyl, morpholinyl or piperazinyl, or

a pharmaceutically acceptable salt thereof.

In embodiments, the compound is a mixture of two compounds as described herein above, often a mixture of two compounds as set forth in FIG. 1 hereof. In embodiments, the compound is a mixture of two compounds selected from the group consisting of compounds 4a,c,d,f-h,j-l; 5b,d,e-l,m; 6a-c; 7a-d,h,i; 8a-f,h-m,o,p; and 9a-c,h,i; 10; 11 and 12 of FIG. 1.

In embodiments, the present invention is directed to pharmaceutical compositions comprising an effective amount of a compound as disclosed above for treating or reducing the likelihood of malaria in a patient or subject in need, in combination with a pharmaceutically acceptable carrier, additive or excipient, optionally in combination with at least one additional bioactive agent, often an additional antimalarial agent. In embodiments, the additional antimalarial agent is a compound, a pharmaceutically acceptable salt thereof or a mixture of compounds which are set forth in FIG. 2, hereof.

In embodiments, the invention is directed to the use of a compound or composition as otherwise described herein for the treatment or prevention (reducing the likelihood) of malaria in a patient or subject in need or at risk for malaria. In embodiments, the method comprises administering a therapeutically effective amount of at least one compound or composition as described herein, optionally in combination with an additional bioactive agent, often an anti-malarial agent to said patient or subject.

In an embodiment, the invention is directed to a method of inhibiting the growth or population of a parasite selected from the group consisting of P. falciparum, P. vivax, P. ovale, P. malariae and P. knowlesi in a patient or subject in need comprising administering a compound or composition as described above to said patient or subject. In embodiments, the parasite is P. falciparum.

Other embodiments of the invention may be readily gleaned from the description of the invention which follows.

### BRIEF DESCRIPTION OF FIGURES

FIG. 1 shows certain preferred compounds of the present invention.

FIG. 2 shows compounds of the prior art which may be used in embodiments for co-administration with compounds according to the present invention (including the preferred compounds of FIG. 1) in pharmaceutical compositions and methods of treating and/or reducing the likelihood of malaria.

FIG. 3 shows the conception of compounds of the present invention as spiroindolone-naphthylisoquinoline hybrids.

FIG. 4 shows representative structurally simple tetrahydroisoquinoline-based compounds.

FIG. 5 shows dot plot showing ranking of DSIIQs in the primary screening against P. falciparum 3D7.

FIG. 6 shows dose-response curves of primary hits against CQ-sensitive P. falciparum 3D7.

FIG. 7 shows dose-response curves of primary hit (8b) against CQ-resistant Pf Dd2 parasite strain.

FIG. 8 shows the effect of (±)-moxiquindole on ring-to-trophozoite development.

FIG. 9 shows the effect of (±)-moxiquindole on trophozoite-to-schizont development.

FIG. 10 shows the effect of (±)-moxiquindole on schizont rupture.

FIG. 11 shows Giemsa-stained thin smears of drug-treated parasites showing the presence of vacuoles within the parasite. ER: Early ring, MT: Mid-trophozoite, MS: Mid-schizont.

FIG. 12 shows the effect of selected antimalarial agents on hemoglobin uptake and hydrolysis in Plasmodium falciparum rings and trophozoites.

FIG. 13 shows the effect of (±)-moxiquindole on vacuolar lipid dynamics in P. falciparum.

FIG. 14/TABLE 1 shows drug-drug interaction studies of (±)-moxiquindole vs chloroquine/artemisinin.

FIG. 15 shows isobologram plots of drug-drug interactions between (±)-moxiquindole and chloroquine/artemisinin. A concave curve represents a synergistic interaction, a convex is consistent with an antagonistic interaction and a straight line is consistent with an additive interaction. Axes are EC50s normalized to 1.

FIG. 16 shows the effect of (±)-moxiquindole on mammalian cells.

FIG. 17 shows the structures of compounds obtained from hit expansion studies.

FIG. 18 shows the antiplasmodial activity of compounds obtained from hit expansion studies.

FIG. 19 shows comparative studies on the effect of (±)-Homoquindole and (±)-Moxiquindole on ring-to-trophozoite development.

FIG. 20 shows comparative studies on the effect of (±)-Homoquindole and (±)-Moxiquindole on trophozoite-to-schizont development.

FIG. 21 shows comparative studies on the effect of (±)-Homoquindole and (±)-Moxiquindole on schizont development.

FIG. 22 shows Giemsa-stained thin smears of drug-treated parasites showing vacuolation in NITD 609-treated and DSIIQ-treated parasites. ER: Early ring, ET: Early trophozoite, ES: Early schizonts.

FIG. 23 shows the effect of DSIIQs and NITD609 on hemoglobin metabolism in the parasite.

FIG. 24/TABLE 2 shows the in silico ADMET properties of selected compounds using PKCMS web too.

FIG. 25/TABLE 1 shows compound drug-likeness and lead-likeness prediction by SWISSADME.

FIG. 26A/TABLE 2 shows the percent chemosuppression of parasitemia by (±)-moxiquindole and (±)-homoquindole in mice infected with P. chabaudi

FIG. 26B shows the chemosuppressive activity of (±)-Moxiquindole and (±)-Homoquindole in Swiss albino mice.

FIG. 27 shows the curative effect of (±)-moxiquindole and (±)-homoquindole against established P. chabaudi infection in Swiss Albino mice.

FIG. 28 shows the survival time of Swiss albino mice treated with (±)-moxiquindole and (±)-homoquindole during P. chabaudi infection.

FIG. 29 shows the effect of α-methylation on the structural similarity between DSIIQs, spiroindolones (NITD609) and NIQs (dioncophylline C).

### DETAILED DESCRIPTION OF THE INVENTION

The following terms as set forth hereinbelow shall be used throughout the specification to describe the present invention. Where a term is not specifically defined herein, that term shall be understood to be used in a manner consistent with its use by those of ordinary skill in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges that may independently be included in the smaller ranges are also encompassed within the invention, subject to any specifically excluded limit in the stated range. Where the stated range includes one or both of the limits, ranges excluding either both of those included limits are also included in the invention. In instances where a substituent is a possibility in one or more Markush groups, it is understood that only those substituents which form stable bonds are to be used.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Furthermore, the following terms shall have the definitions set out below.

The term “patient” or “subject” is used throughout the specification within context to describe an animal, generally a mammal, especially and preferably a human, to whom treatment, including prophylactic treatment (prophylaxis), with the compounds or compositions according to the present invention is provided. For treatment of those infections, conditions or disease states which are specific for a specific animal such as a human patient, the term patient refers to that specific animal. In most instances, the patient or subject of the present invention is a human patient of either or both genders.

The term “effective” is used herein, unless otherwise indicated, to describe an amount of a compound or component which, when used within the context of its use, produces or effects an intended result in the treatment of malaria, whether that result relates to the prophylaxis and/or therapy of an infection and/or disease state within the context of its use or as otherwise described herein. The term effective subsumes all other effective amount or effective concentration terms (including the term “therapeutically effective”) which are otherwise described or used in the present application.

The term “compound” is used herein to describe any specific compound or bioactive agent disclosed herein, including any and all stereoisomers (including diastereomers, individual optical isomers/enantiomers or racemic mixtures and geometric isomers), pharmaceutically acceptable salts and prodrug forms. The term compound herein refers to stable compounds. Within its use in context, the term compound may refer to a single compound or a mixture of compounds as otherwise described herein. It is understood that the choice of substituents or bonds within a Markush or other group of substituents or bonds is provided to form a stable compound from those choices within that Markush or other group.

The term “pharmaceutically acceptable” as used herein means that the compound or composition is suitable for administration to a subject to achieve the treatments described herein, without unduly deleterious side effects in light of the severity of the disease and necessity of the treatment.

“Alkyl” refers to a fully saturated monovalent radical containing carbon and hydrogen, and which may be cyclic, branched or a straight chain. Examples of alkyl groups are methyl, ethyl, n-butyl, n-hexyl, n-heptyl, n-octyl, n-nonyl, n-decyl, isopropyl, 2-methyl-propyl, cyclopropyl, cyclopropylmethyl, cyclobutyl, cyclopentyl, cyclopentylethyl, cyclohexylethyl and cyclohexyl. Preferred alkyl groups are C₀-C₆ alkyl groups (which includes C₀ as H). Even more preferred alkyl groups are C₁-C₆ alkyl groups. “Alkylene” refers to a fully saturated hydrocarbon which is divalent (may be linear, branched or cyclic) and which is optionally substituted. Preferred alkylene groups are C₁-C₆ alkylene groups. Other terms used to indicate substituent groups in compounds according to the present invention are as conventionally used in the art.

“Alkylene” refers to a fully saturated hydrocarbon which is divalent (may be linear, branched or cyclic) and which is optionally substituted. Other terms used to indicate substituent groups in compounds according to the present invention are as conventionally used in the art. Thus, the term alkylene aryl includes alkylene phenyl such as a benzyl group or ethylene phenyl group, alkylaryl, includes alkylphenyl such a phenyl group which has alkyl groups as substituents, etc. The bond [symbol] , when used in chemical structures of the present application refers to a single chemical bond, which may be an optional double bond, in context.

The term “aryl” or “aromatic”, in context, refers to a substituted or unsubstituted monovalent aromatic radical having a single ring (e.g., benzene or phenyl) or fused rings (naphthyl). Aromatic heterocycles (which contain 1, 2, 3 or 4 atoms other than carbon (e.g. nitrogen, sulfur, oxygen, phosphorous or other atoms are heteroaryls in the present application.

The term “heterocycle” or “heterocyclic” shall mean an optionally substituted moiety that is cyclic and contains at least one atom other than a carbon atom, such as a nitrogen, sulfur, oxygen or other atom. A heterocyclic ring shall contain up to four atoms other than carbon selected from nitrogen, sulfur and oxygen. These rings may be saturated or have unsaturated bonds. As otherwise described, aromatic heterocycles are heteroaryls. Fused rings are also contemplated by the present invention. A heterocycle according to the present invention is an optionally substituted imidazole, a piperazine (including piperazinone), piperidine, furan, pyrrole, imidazole, thiazole, oxazole or isoxazole group, among numerous others. Depending upon its use in context, a heterocyclic ring may be saturated and/or unsaturated.

“Alkoxy” as used herein refers to an alkyl group bound through an ether linkage; that is, an “alkoxy” group may be represented as —O-alkyl where alkyl is as defined above.

“Hydrocarbon” or “hydrocarbyl” refers to any radical containing carbon and hydrogen, which may be straight, branch-chained or cyclic in nature. Hydrocarbons include linear, branched and cyclic hydrocarbons, including alkyl groups, alkylene groups and unsaturated hydrocarbon groups, which may be optionally substituted. Hydrocarbyl groups may be fully saturated or unsaturated, containing one or more double (“ene”) or triple (“yne”) bonds.

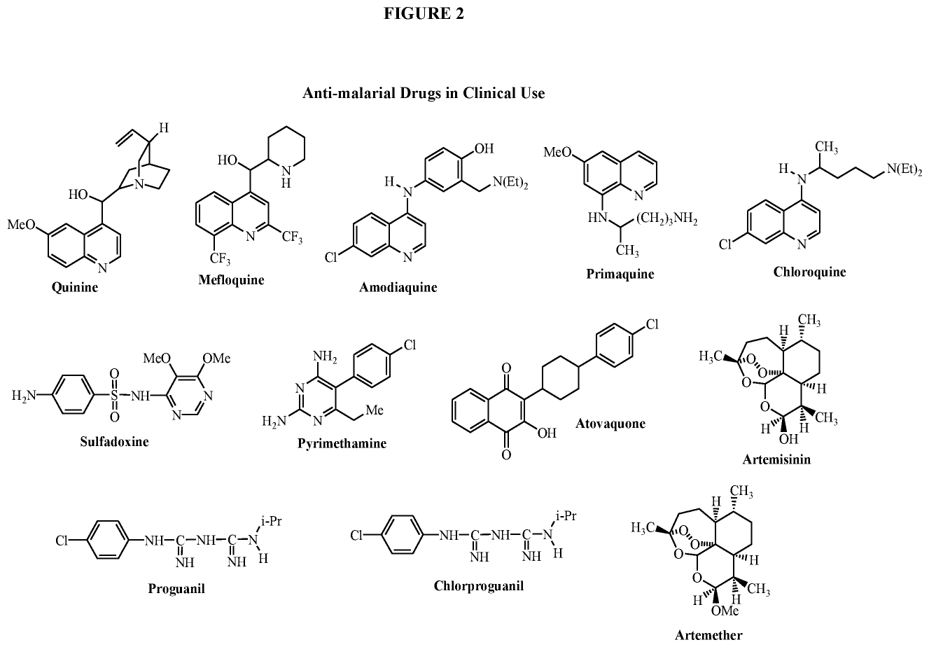

The term “bioactive agent” refers to any biologically active compound or drug which may be formulated for use in the present invention. Exemplary bioactive agents include the compounds according to the present invention which are used to treat malaria as well as other disease states and/or conditions which are otherwise described herein. Preferred exemplary additional anti-malarial compounds for co-administration with compounds according to the present invention include the compounds which are presented in FIG. 2 hereof.

The terms “treat”, “treating”, and “treatment”, are used synonymously to refer to any action providing a benefit to a patient at risk for or afflicted with a disease, including improvement in the condition through lessening or suppression of at least one symptom, delay in progression of the disease or delay in the onset of the disease, etc. Treatment, as used herein, encompasses prophylactic and therapeutic treatment, depending on the context of the treatment used. Compounds according to the present invention can, for example, be administered prophylactically to a mammal in advance of the occurrence of disease to inhibit or reduce the likelihood of that disease. Prophylactic administration is effective to reduce or decrease the likelihood of the subsequent occurrence of disease in the mammal or decrease the severity of disease that subsequently occurs. Alternatively, compounds according to the present invention can, for example, be administered therapeutically to a mammal that is already afflicted by disease. In one embodiment of therapeutic administration, administration of the present compounds is effective to eliminate the disease and produce a remission or substantially eliminate (i.e. cure) the causative agent (parasite), symptoms of a disease state and/or condition; in another embodiment, administration of the compounds according to the present invention is effective to decrease the severity of the disease or lengthen the lifespan of the mammal so afflicted, in the case of malaria. In embodiments, the present invention is directed to methods of treatment of malaria.

The term “pharmaceutically acceptable” as used herein means that the compound or composition is suitable for administration to a subject to achieve the treatments described herein, without unduly deleterious side effects in light of the severity of the disease and necessity of the treatment.

The term “inhibit” as used herein refers to the partial or complete elimination of a potential effect such as a symptom or a secondary condition of a disease state, while inhibitors are compounds that have the ability to inhibit.

The term “prevention” when used in context shall mean “reducing the likelihood” or preventing a condition or disease state from occurring as a consequence of administration or concurrent administration of one or more compounds or compositions according to the present invention, alone or in combination with another agent. It is noted that prophylaxis will rarely be 100% effective; consequently the terms prevention and reducing the likelihood are used to denote the fact that within a given population of patients of subjects, administration with compounds according to the present invention will reduce the likelihood or inhibit a particular condition or disease state (in particular, the worsening of a disease state such as malaria or other accepted indicators of disease progression from occurring.

The term “malaria” shall refer to Malaria is a mosquito-borne disease caused by a parasite. People with malaria often experience fever, chills, and flu-like illness. Left untreated, they may develop severe complications and die. In 2018 an estimated 228 million cases of malaria occurred worldwide and 405,000 people died, mostly children in the African Region. About 2,000 cases of malaria are diagnosed in the United States each year. The vast majority of cases in the United States are in travelers and immigrants returning from countries where malaria transmission occurs, many from sub-Saharan Africa and South Asia. Malaria is a serious and sometimes fatal disease caused by a parasite that commonly infects a certain type of mosquito which feeds on humans. People who get malaria are typically very sick with high fevers, shaking chills, and flu-like illness. Although malaria can be a deadly disease, illness and death from malaria can usually be prevented. About 2,000 cases of malaria are diagnosed in the United States each year. The vast majority of cases in the United States are in travelers and immigrants returning from countries where malaria transmission occurs, many from sub-Saharan Africa and South Asia.

The natural history of malaria involves cyclical infection of humans and female Anopheles mosquitoes. In humans, the parasites grow and multiply first in the liver cells and then in the red cells of the blood. In the blood, successive broods of parasites grow inside the red cells and destroy them, releasing daughter parasites (“merozoites”) that continue the cycle by invading other red cells.

Infection of the human host by the parasite causes fever and chills, anemia and death if left untreated. There are several species of the parasite; however, the most common five that infect humans are P. falciparum, P. vivax, P. ovale, P. malariae and P. knowlesi. Among the five, P. falciparum is responsible for the bulk of malaria cases. The life cycle of Plasmodium involves two hosts, the female Anopheles mosquito (which also serves as vector) and the human, and it comprises several stages (reviewed in Turner, 2016). When an infected female Anopheles mosquito bites a human and takes a blood meal, it also injects into the bloodstream of the human the sporozoite stage of Plasmodium. These sporozoites travel to the liver where they infect the hepatocytes and mature into schizonts. The schizonts later burst and release merozoites into the blood stream. Within the liver, the sporozoites may also go into dormancy resulting in hypnozoites; the reactivation of the latter is responsible for relapse of malaria. The merozoites which enter the bloodstream infect erythrocytes and transform into rings, trophozoites and finally into schizonts. The latter burst and release more merozoites into the bloodstream thereby propagating the infection. A small fraction of merozoites undergoes transformation into gametocytes that are subsequently ingested by the mosquito when it takes a blood meal. Within the mosquito gut, these gametocytes undergo sexual reproduction to eventually produce the sporozoites which migrate to the salivary gland of the mosquito and are injected into the human host to complete the cycle. The complex life cycle of Plasmodium provides a number of targets for intervention by chemotherapeutic agents.

The blood stage parasites are those that cause the symptoms of malaria. When certain forms of blood stage parasites (gametocytes, which occur in male and female forms) are ingested during blood feeding by a female Anopheles mosquito, they mate in the gut of the mosquito and begin a cycle of growth and multiplication in the mosquito. After 10-18 days, a form of the parasite called a sporozoite migrates to the mosquito's salivary glands. When the Anopheles mosquito takes a blood meal on another human, anticoagulant saliva is injected together with the sporozoites, which migrate to the liver, thereby beginning a new cycle.

Thus the infected mosquito carries the disease from one human to another (acting as a “vector”), while infected humans transmit the parasite to the mosquito, In contrast to the human host, the mosquito vector does not suffer from the presence of the parasites. The malaria parasite life cycle involves two hosts. During a blood meal, a malaria-infected female Anopheles mosquito inoculates sporozoites into the human host. Sporozoites infect liver cells and mature into schizonts, which rupture and release merozoites. (Of note, in P. vivax and P. ovale a dormant stage [hypnozoites] can persist in the liver (if untreated) and cause relapses by invading the bloodstream weeks, or even years later.) After this initial replication in the liver (exo-erythrocytic schizogony), the parasites undergo asexual multiplication in the erythrocytes (erythrocytic schizogony). Merozoites infect red blood cells. The ring stage trophozoites mature into schizonts, which rupture releasing merozoites. Some parasites differentiate into sexual erythrocytic stages (gametocytes). Blood stage parasites are responsible for the clinical manifestations of the disease. The gametocytes, male (microgametocytes) and female (macrogametocytes), are ingested by an Anopheles mosquito during a blood meal. The parasites' multiplication in the mosquito is known as the sporogonic cycle. While in the mosquito's stomach, the microgametes penetrate the macrogametes generating zygotes. The zygotes in turn become motile and elongated (ookinetes) which invade the midgut wall of the mosquito where they develop into oocysts. The oocysts grow, rupture, and release sporozoites, which make their way to the mosquito's salivary glands. Inoculation of the sporozoites into a new human host perpetuates the malaria life cycle.

The term “additional anti-malaria agent” is used to describe an additional compound which may be co-administered with one or more compounds of the present invention in the treatment of malaria. These compounds, among others are identified in FIG. 2 hereof.

The present invention includes the compositions comprising the pharmaceutically acceptable salt. i.e., the acid or base addition salts of compounds of the present invention and their derivatives. The acids which may be used to prepare the pharmaceutically acceptable acid addition salts of the aforementioned base compounds useful in this invention are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)]salts, among others.

Pharmaceutically acceptable base addition salts may also be used to produce pharmaceutically acceptable salt forms of the compounds according to the present invention. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (eg., potassium and sodium) and alkaline earth metal cations (e, calcium and magnesium), ammonium or water-soluble amine addition salts such as N-methylglucamine-(meglumine), and the lower alkanolammonium and other base salts of pharmaceutically acceptable organic amines, among others.

Compounds according to the present invention may be readily formulated into pharmaceutical compositions, useful in the treatment of disease states and/or conditions as otherwise described herein. These disease states and/or conditions principally include malaria.

Pharmaceutical compositions comprise an effective amount of one or more compounds according to the present invention in combination with a pharmaceutically acceptable carrier, additive or excipient, optionally in combination with at least one additional anticancer agent.

As noted above, the compounds and method of the invention are useful for the inhibition (including prophylaxis) and/or treatment of malaria. In methods according to the present invention, subjects or patients in need are treated with effective amounts of the present compounds, pharmaceutical compositions in order to inhibit, reduce the likelihood or treat a disease state, condition and/or infection as otherwise described herein. The disease states, conditions and infections treated by the present compounds and compositions are readily recognized and diagnosed by those of ordinary skill in the art and treated by administering to the patient an effective amount of one or more compounds according to the present invention.

Regardless of the mechanism, the compounds of the present invention may be used to treat disease states or conditions in patients or subjects who suffer from those conditions or disease states or are at risk for those conditions. In this method a compound in an effective amount is administered to a patient in need of therapy to treat the condition(s) or disease state(s). These disease states or conditions are directed principally to malaria.

Generally, dosages and routes of administration of the compound are determined according to the size and condition of the subject, according to standard pharmaceutical practices. Dose levels employed can vary widely, and can readily be determined by those of skill in the art. Typically, amounts in the milligram up to gram quantities are employed. The composition may be administered to a subject by various routes, e.g. orally, transdermally, perineurally or parenterally, that is, by intravenous, subcutaneous, intraperitoneal, or intramuscular injection, among others, including buccal, rectal, and transdermal administration. Subjects contemplated for treatment according to the method of the invention include humans, companion animals, laboratory animals, and the like.

Formulations containing the compounds according to the present invention may take the form of solid, semi-solid, lyophilized powder, or liquid dosage forms, such as, for example, tablets, capsules, powders, sustained-release formulations, solutions, suspensions, emulsions, suppositories, creams, ointments, lotions, aerosols, patches or the like, preferably in unit dosage forms suitable for simple administration of precise dosages.

Pharmaceutical compositions according to the present invention typically include a conventional pharmaceutical carrier or excipient and may additionally include other medicinal agents, carriers, adjuvants, additives and the like. Often, the composition is about 0.1% to about 85%, about 0.5% to about 75% by weight of a compound or compounds of the invention, with the remainder consisting essentially of suitable pharmaceutical excipients. For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

Liquid compositions can be prepared by dissolving or dispersing the compounds (about 0.5% to about 20% by weight or more), and optional pharmaceutical adjuvants, in a carrier, such as, for example, aqueous saline, aqueous dextrose, glycerol, or ethanol, to form a solution or suspension. For use in oral liquid preparation, the composition may be prepared as a solution, suspension, emulsion, or syrup, being supplied either in liquid form or a dried form suitable for hydration in water or normal saline.

When the composition is employed in the form of solid preparations for oral administration, the preparations may be tablets, granules, powders, capsules or the like. In a tablet formulation, the composition is typically formulated with additives, e.g. an excipient such as a saccharide or cellulose preparation, a binder such as starch paste or methyl cellulose, a filler, a disintegrator, and other additives typically used in the manufacture of medical preparations.

An injectable composition for parenteral administration will typically contain the compound in a suitable i.v. solution, such as sterile physiological salt solution. The composition may also be formulated as a suspension in a lipid or phospholipid, in a liposomal suspension, or in an aqueous emulsion.

Methods for preparing such dosage forms are known or are apparent to those skilled in the art; for example, see Remington's Pharmaceutical Sciences (17th Ed., Mack Pub. Co., 1985). The composition to be administered will contain a quantity of the selected compound in a pharmaceutically effective amount for treating malaria according to the present invention in a subject.

### Examples

Chemotherapy of Malaria (Antimalarial Agents)

Antimalarial agents in clinical use and under investigation belong to a number of chemical classes which are described below (reviewed in Biamonte et al., 2013; Ashton et al., 2019; Tse et al., 2019). Commonly used antimalarial drugs are set forth in FIG. 2 hereof.

Quinolines:

Quinine, probably one of the oldest antimalarial agents, and its analogues and derivatives are among some of the oldest antimalarial agents. Isolated from the bark of the South American tree Cinchona sp, the compound quickly became the standard treatment for malaria in Europe in the early 17^{th} century. Chemical modification of quinine eventually gave rise to several quinoline-based compounds that have found clinical use in malaria treatment over the years, including chloroquine, mefloquine, amodiaquine, quinacrine, mepacrine. A common feature of these compounds is that they interfere with the conversion of toxic heme to crystalline hemozoin, an essential process for the survival of the parasite.

Folate Analogues and derivatives: These include pyrimethamine, dapsone, sulfadoxine, proguanil, cycloguanil. These compounds interfere with folate metabolism either at the level of dihydrofolate reductase, dihydropteroate synthetase or dihydroorotate synthetase and have shown varying degrees of effectiveness against malaria over time.

Peroxides: Since the 1980s, this class of antimalarials has grown very rapidly inspired by the impressive antimalarial activity of the naturally occurring endoperoxide artemisinin. Compounds in this class now include artesunate, artemether, and other synthetic endoperoxides such as OZ277. Largely due their short half-lives, and to forestall the emergence of resistance to this class of antimalarials, the peroxides are now administered in combination with a longer lived partner drug to reduce recrudescence. Termed Artemisinin Combination Therapy (ACT), this strategy has become the standard therapy for uncomplicated malaria.

Other Compounds: These include atovaquone, a mitochondrial electron transport inhibitor and halofantrine.

Antibiotics: A number of antibiotics, including fosmidomycin and clindamycin, display antimalarial activity.

Many of the drugs belonging to the above-named chemical classes have recorded many successes over the years. However, several of them have been eventually rendered ineffective in certain parts of the world due to the emergence of drug resistance. To combat resistance, a number of drug combinations have been introduced into the market. However, even some drug combinations like pyrimethamine/sulfadoxine have been compromised when resistance develops to one partner (reviewed in World Malaria Report 2018; Mathews & John, 2018). Recent reports of emerging resistance to some ACTs have also put in doubt the durability of this strategy and highlighted the need to employ new strategies in antimalarial drug discovery. One such strategy, termed molecular hybridization, is the covalent linking of the pharmacophoric elements of two or more bioactive molecules into one molecular scaffold in order to produce a multi-target drug (reviewed in Pedrosa et al., 2017; Tibon et al., 2020). Multi-target agents are expected to be more efficient than their single target counterparts because they inhibit multiple biological processes in the parasite and can therefore induce massive fatal disruption of parasite function and loss of viability. Moreover, these agents are less prone to the emergence of resistance than single target agents as it would take more than one genetic mutation to reverse the widespread dysfunction precipitated by these agents. Finally, multi-target agents can eliminate problems encountered with discordant pharmacokinetic profiles of partners in drug combinations.

### Antimalarial Discovery by Combination of Antimalarial Chemotypes: 3′,4′-Dihydro-2′H-spiro[indoline-3:1′-isoquinolin]-2-ones as Spiroindolone-Naphthylisoquinoline Hybrids

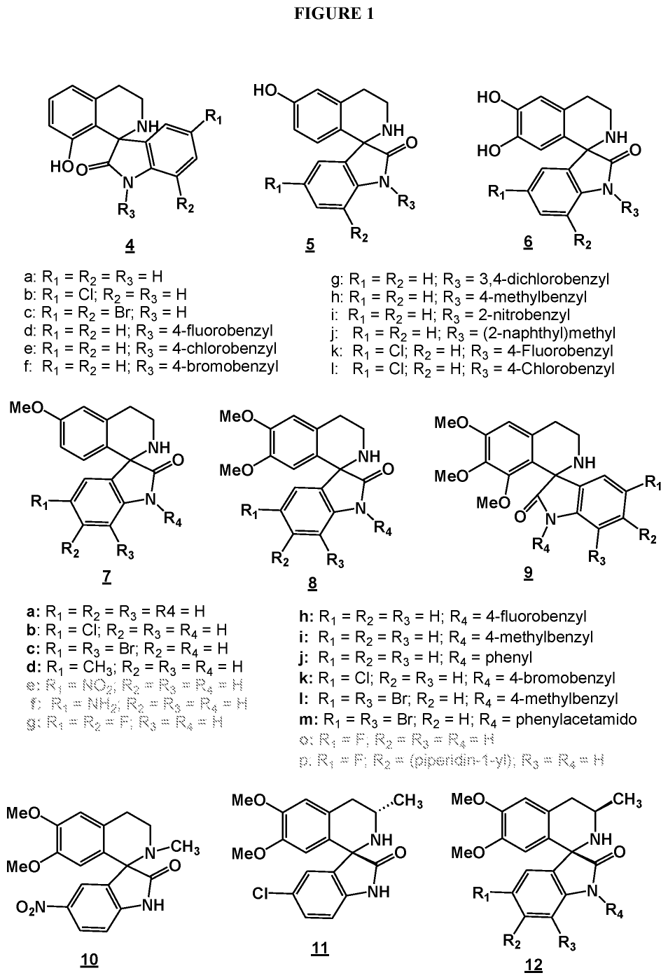

A recent communication (Lobe & Efange, 2020) describes the design, synthesis and antiproliferative activity of several analogues of 3′,4′-dihydro-2′H-spiro[indoline-3:1′-isoquinolin]-2-one (DSIIQ) (FIG. 1), a previously unexplored class of spirooxindoles.

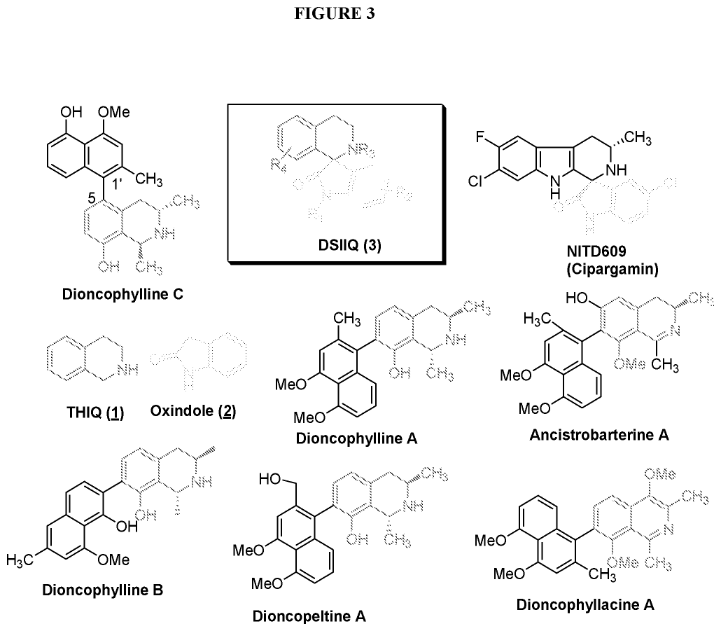

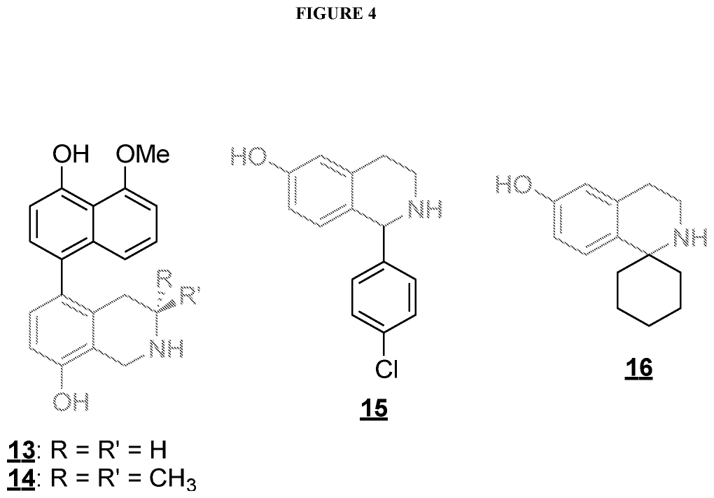

However, the following analysis identified these compounds as spiroindolone-naphthylisoquinoline hybrids and therefore prompted their screening for antimalarial activity:
1) DSIIQs (3) and the spiroindolones (exemplified by NITD609), have one common structural feature: the oxindole fragment 2 (see FIG. 3). In recent years, the spiroindolones have emerged as a promising new class of antimalarials with a novel mechanism of action. Compounds in this class, represented by KAE609 (NITD609, cipargamin) which is currently in clinical trials, inhibit Pf ATP-4 and thus trigger a catastrophic disruption of sodium ion homeostasis. As a result, NITD609 inhibits all erythrocytic stages of the P. falciparum life cycle and transmission of the parasite (Rottmann et al., 2010; Yeung et al., 2010; Turner, 2016). Structurally, both DSIIQs and spiroindolones are 3,3-disubstituted oxindoles; however, in the spiroindolones, the oxindole fragment is fused to the THβC scaffold while in DSIIQ the oxindole fragment is fused to the THIQ fragment. The spiroindolones may thus be regarded as THβC-substituted oxindoles while the DSIIQs may be viewed as THIQ-substituted oxindoles.
2) DSIIQs and naphthylisoquinolines (NIQs) (exemplified by dioncophylline C, dioncophylline A and other naphthylisoquinolines) also have one common structural feature: the tetrahydroisoquinoline (THIQ) fragment (1, FIG. 3). The NIQs belong to two plant families, Dioncophyllaceae and Ancistrocladaceae, that are found mostly in Africa and Southeast Asia. This class of structurally diverse secondary metabolites is characterized by the presence of a THIQ scaffold substituted with a naphthyl group. The NIQs are classified by the position of the THIQ-naphthalene or biaryl axis: 5-1′, 5-3′, 5-8′, 7-1′, 7-3′, 7-6′,7-8′ and C—N. The NIQs display remarkable anti-malarial, anti-trypanosomal, anti-leishmanial, fungicidal, molluscicidal, larvicidal, insecticidal, spasmolytic, and anti-HIV activities (reviewed in Ibrahim & Mohammed, 2015). Several NIQs have been reported to display potent activity against the asexual erythrocytic stages of P. falciparum and P. berghei in vitro (Li et al., 2017; Fayez et al., 2018) and some, like dioncophylline C and dioncopeltine A, were reported to be curative in vivo (Francois et al., 1997a). In a study of NIQs isolated from the Dioncophyllaceae and Acistroacladaceae, three compounds, dioncophylline A, dioncophyllacine A and ancistroberterine A emerged as active against the liver stages of the P. berghei (Francois et al., 1997b). The first two compounds were more active than primaquine in the same test system, suggesting that NIQs are active on both asexual erythrocytic and exoerythrocytic stages of the Plasmodium life cycle. In another study of the effect of dioncophylline B on the erythrocytic stages of the malaria parasite P. chabaudi, it was observed that the ring stages are insensitive to the compound while the trophozoites are quite sensitive and the schizonts are only partially sensitive to the compound (Francois et al., 1999). This preliminary evidence suggests that these compounds may target hemoglobin degradation (Francois et al., 1999). Recent studies have confirmed that the NIQs are multi-stage active antimalarials (Moyo et al., 2020). As a result of their remarkable pharmacological profile NIQs have been proposed as potentially useful lead compounds for malaria drug discovery.
3) The THIQ, L fragment is the antimalarial pharmacophore in the NIQs. In a previous study of 5-naphthyltetrahydroisoquinolines and -dihydroisoquinolines, Bringmann et al. (2010) showed that structurally simplified THIQs, such as 13 and 14 (See FIG. 4), are highly active against P. falciparum. A subsequent report of 6-hydroxy-1-phenyl- and 6-hydroxy-1,1-spirofused THIQs from our group also showed that structurally simple THIQ analogues like 15 and 16, respectively (FIG. 4), display antimalarial activity comparable to that of chloroquine (Ngo Hanna et al., 2014). These findings clearly suggest 1) that the antimalarial pharmacophore of the NIQs is found within the THIQ scaffold; and 2) that 1,1-spirofused THIQs can display antiplasmodial activity. Consequently, it was reasonable to propose that the DSIIQs, which contain this pharmacophore and which are 1,1-spirofused would also possess antimalarial properties.

At its core, the present invention is thus based on the recognition that the DSIIQ scaffold incorporates pharmacophoric elements of two mechanistically dissimilar antimalarial scaffolds, spiroindolones and NIQs, and can therefore be considered a spiroindolone-naphthylisoquinoline hybrid. Consequently, the DSIIQs were expected to yield compounds that display antiplasmodial activity and possibly combine the modes of action of both the NIQs and spiroindolones to become multi-target antimalarial agents.

The representative compounds of this invention found in FIG. 1 were therefore screened for antiplasmodial activity. The synthesis of these compounds has been reported (Lobe & Efange, 2020). Previously unreported analogues are described herein.

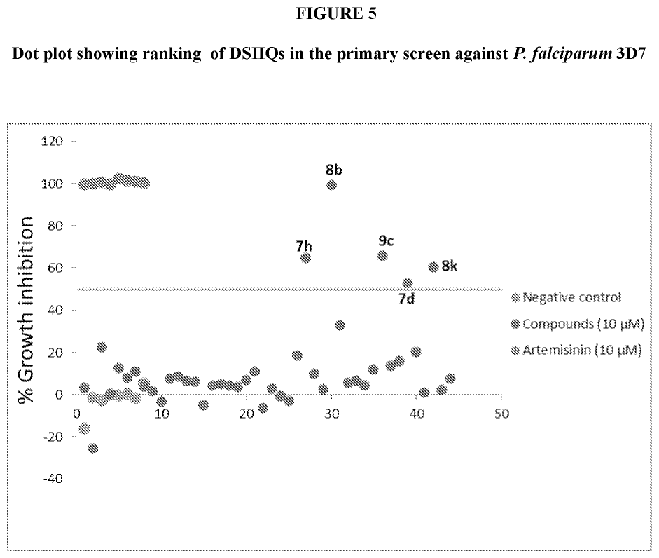

In an effort to identify bioactive DSIIQs, a library of 45 analogues was tested against the CQ-sensitive 3D7 strain of P. falciparum, using a two-tier system (Bennet et al., 2004). The SYBR green I-based parasite growth inhibition assay was used to assess antiplasmodial activities, and hits exhibiting greater than 50% parasite growth inhibition in primary assays were selected and subjected to dose-response hit confirmation analyses. In all, 5 primary hits (7d, 7h, 8b, 8k, 9c) were identified with growth inhibitory activities of approximately 59%, 64%, 97%, 52%, 65%, respectively (see FIG. 5).

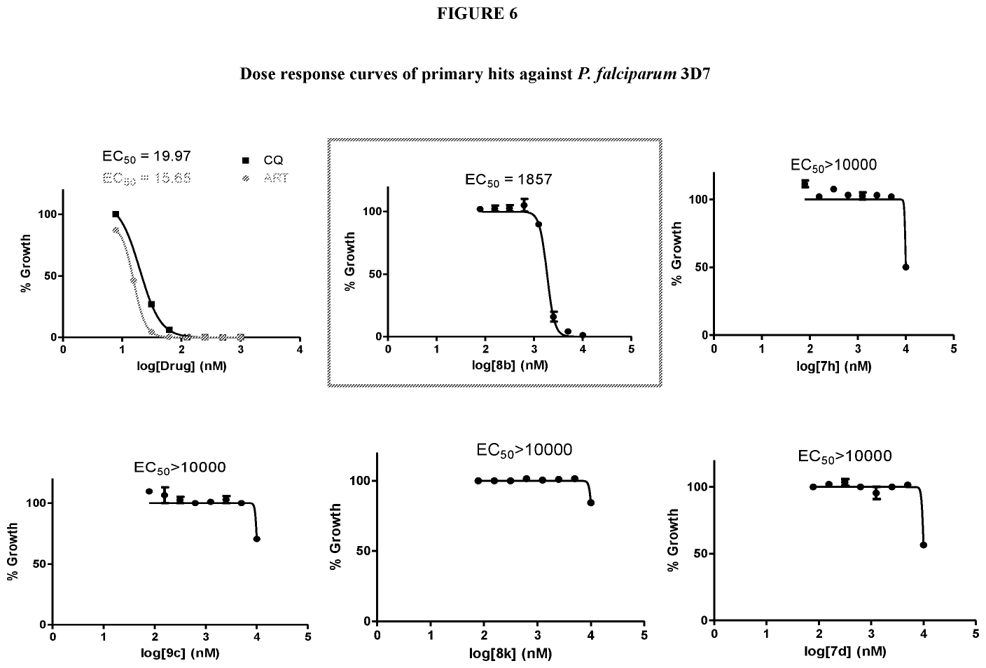

Primary hits were subsequently tested against the CQ-sensitive Pf 3D7 strain across a concentration range of 0.078 μM to 10 μM to assess the dose dependence of the measured activities. As shown in FIG. 6, only one compound, (8b), subsequently named (±)-moxiquindole, produced a complete sigmoidal dose-response curve within the tested drug concentration range with an EC₅₀<2 μM. The other six primary hits did not produce complete dose-response curves, and as such were considered unsuitable for use in further hit optimization studies. Artemisinin and chloroquine exhibited potent inhibitory activities with EC₅₀ values of 15.88 and 13.88 nM, respectively.

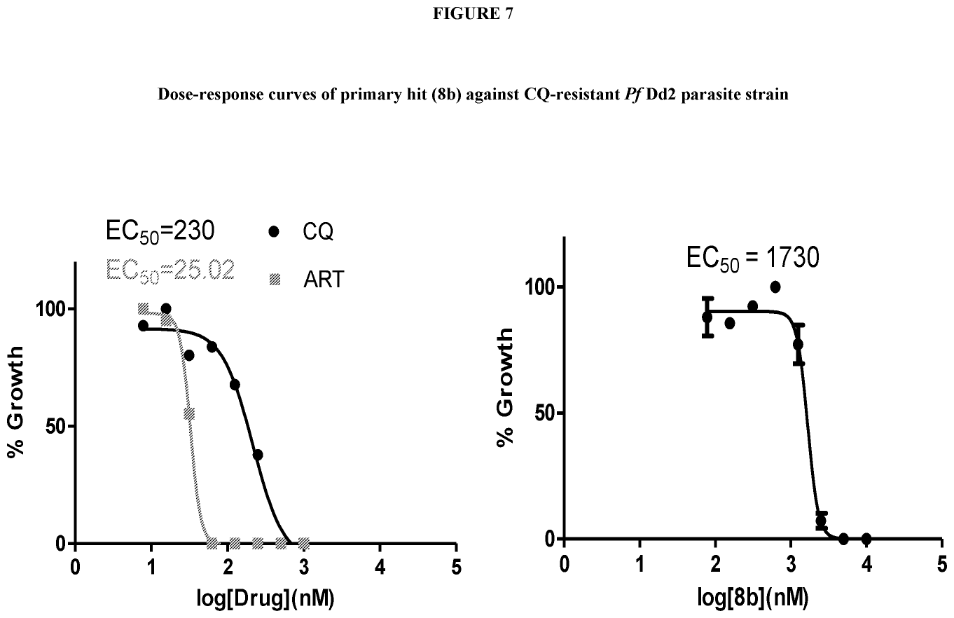

As preliminary assessment of the cross-resistance potential of 8b, the compound was further tested against the multidrug resistant strain Dd2 essentially as described in assays against the chloroquine-resistant 3D7 parasite strain. As shown in FIG. 7, the compound produced a complete dose-response curve within the tested concentration range (0.078 μM to 10 μM) with an EC50 value of 1730 nM. This EC50 value is similar to the EC₅₀ obtained against the 3D7 chloroquine-sensitive strain (cross-resistance index of 0.935), indicating no measurable cross-resistance between the new compound and chloroquine.

The life cycle stage specific action of (±)-moxiquindole was determined by treatment of parasites at different time points followed by microscopic analysis of Giemsa-stained thin blood smears. Stage proportions were calculated for each asexual parasite stage in the drug treated wells relative to the same stage in the negative control wells.

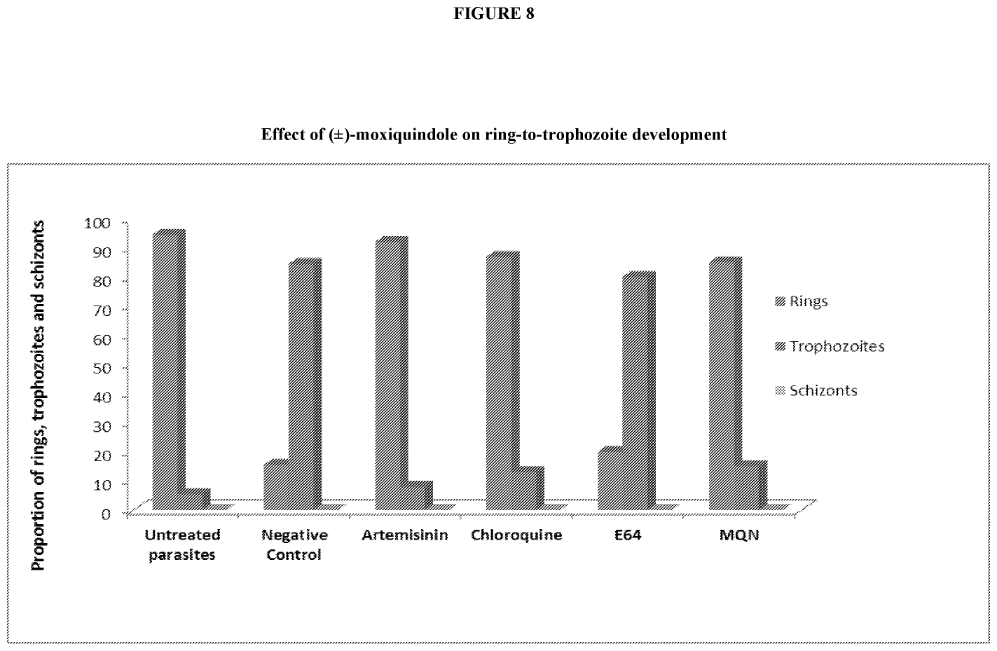

As shown in FIGS. 8, 9 and 10, treatment of parasites at the early ring (ER), mid-trophozoite (MT) or mid-schizont (MS) stages with either artemisinin or the new compound resulted in high proportions of the treated parasite stage at each treatment time-point, indicating the fast-acting activity of both artemisinin and the compound against all major asexual blood stages of P. falciparum. Contrarily, treatment with chloroquine resulted in rapid accumulation of early rings and trophozoites but not schizonts at the respective treatment time-points. Similarly, the cysteine protease inhibitor E64 (L-trans-epoxysuccinyl-leucylamido-(4-guanidino) exhibited high stage specificity, only inhibiting schizont accumulation compared to the new compound that is multi-stage active. Therefore, (±)-moxiquindole differs from both chloroquine and E64.

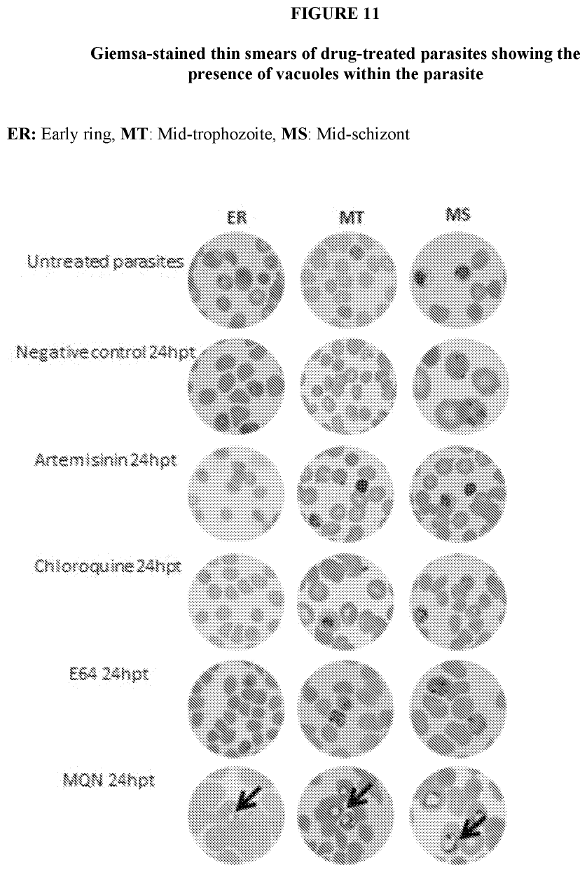

As shown in FIG. 11, unlike what was seen in the chloroquine or artemisinin treated cultures, parasites exposed to (±)-moxiquindole showed characteristic morphological features of dying cells such as abnormal vacuolation, indicating some differences in the antiplasmodial modes of action of the compound compared to ART or CQ. The vacuolation phenotype was common across all parasite stages, and tended to increase in size with parasite maturity (larger in schizonts than in trophozoites and ring stage parasites). This latter observation suggests a possible enrichment of the drug target in late-stage parasites or increase in the drug effect with parasite age.

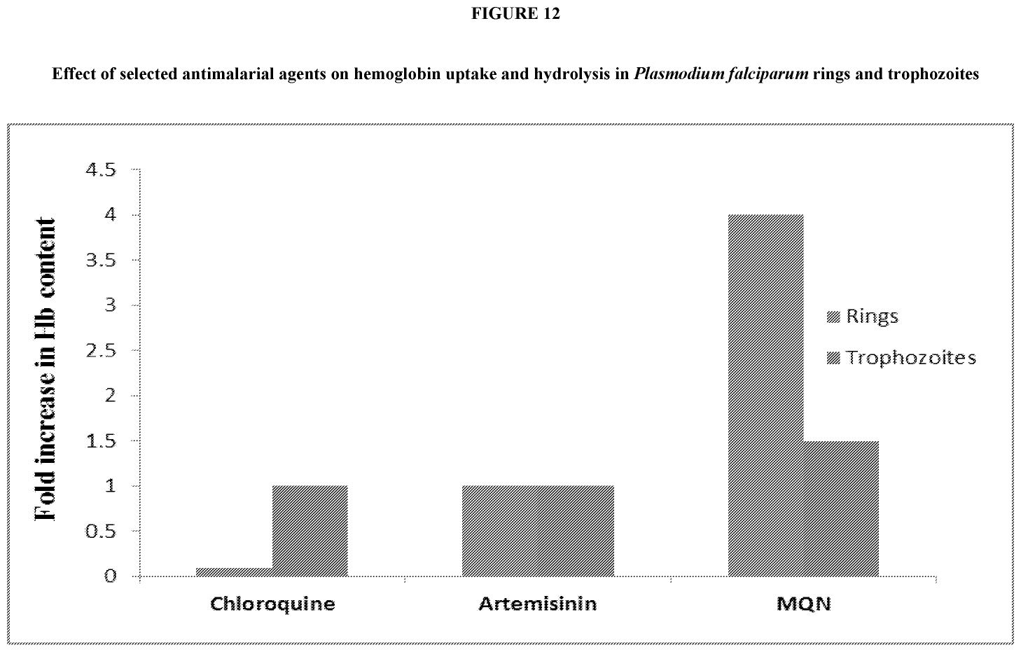

Owing to the fact that the DSIIQs are constructed from the THIQ pharmacophore of naphthylisoquinolines, which appear to target proteases involved in hemoglobin degradation, we assessed the effect of this DSIIQ on hemoglobin metabolism. Briefly, tightly synchronized ring and trophozoite stage parasites were treated with test compounds for 24 h followed by parasite isolation and quantification of the intraparasitic hemoglobin contents. As shown in FIG. 12, the hemoglobin content (expressed as fold increase over E64-treated cultures) was significantly higher in (±)-moxiquindole than in chloroquine or artemisinin-treated cultures. Additionally, the effect of the compound on hemoglobin accumulation was greater when added at the ring-stage than in trophozoite parasites. Together, these findings suggest novel effects of the compound on hemoglobin metabolism distinct from known effects of both chloroquine and artemisinin on heme metabolism.

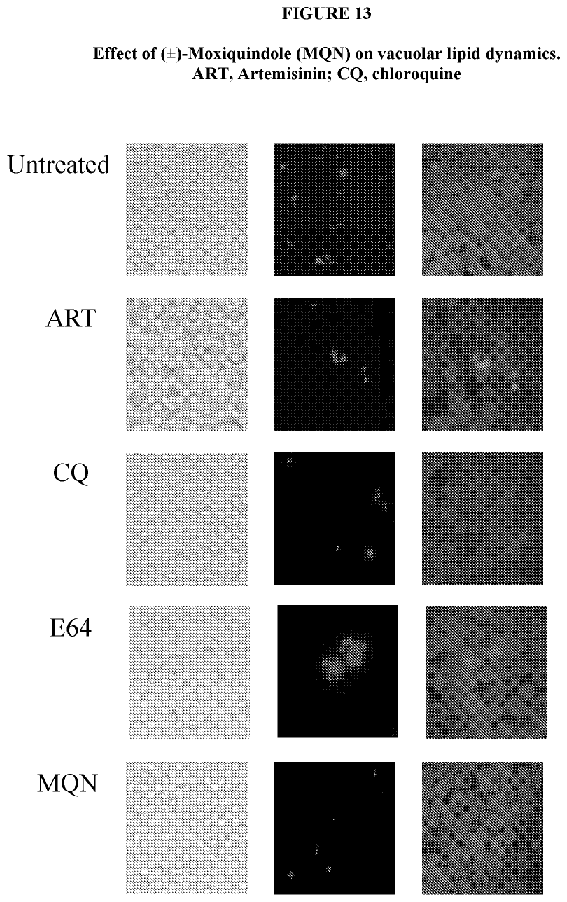

Because cellular vacuolation may result from perturbation of intracellular lipid content, we investigated the effect of (±)-moxiquindole on vacuolar lipid dynamics using fluorescent Oil red O as lipid stain. As shown in FIG. 13, Oil red O distinctly stained intracellular spots in trophozoite and schizont stage parasites in untreated cultures as well as in artemisinin and E-64 treated cultures, but not in chloroquine and (±)-moxiquindole treated parasites. Together, these findings suggest that both chloroquine and (±)-moxiquindole may exhibit antiplasmodial activity by disrupting lipid deposition at intracellular sites proximal to the parasite digestive vacuole.

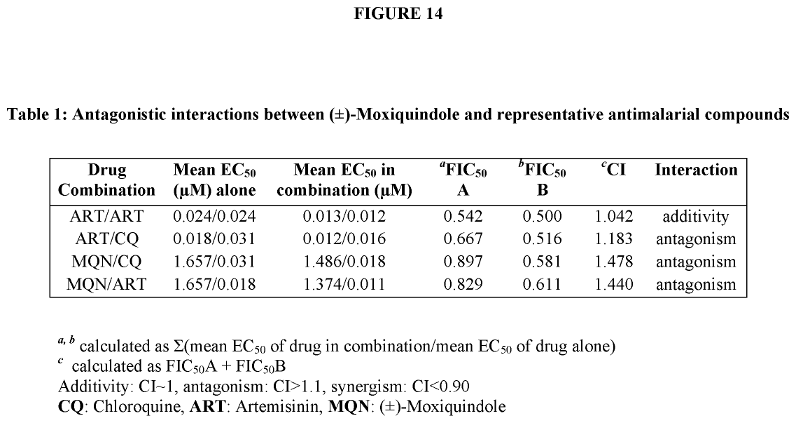

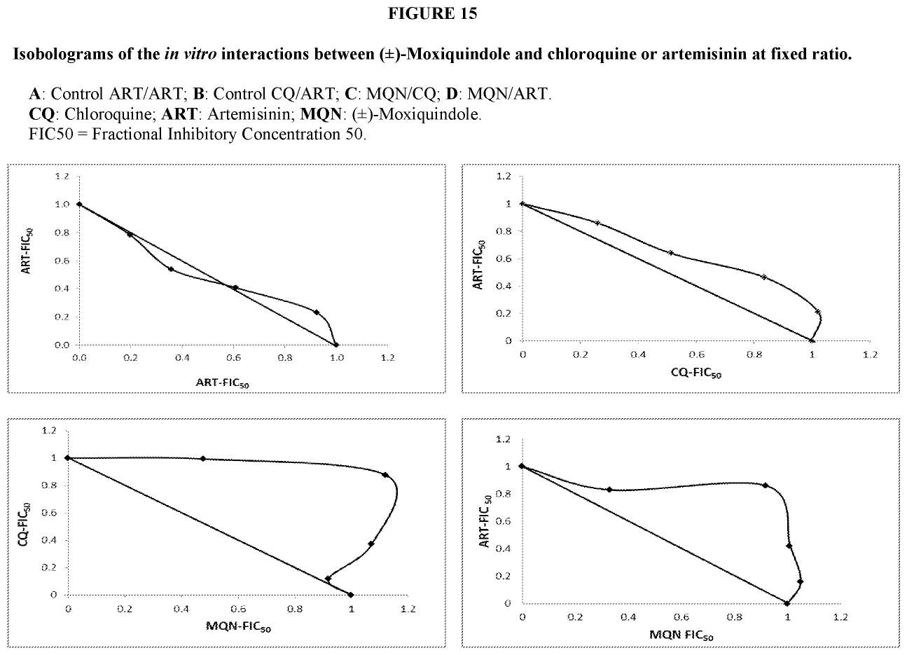

Given the apparent overlapping modes of action between this hybrid and some known antimalarial compounds in terms of targeted cellular processes, we investigated the interaction between these compounds at variable combination ratios. As presented in FIG. 14/Table 1 and FIG. 15, (±)-moxiquindole exhibited antagonistic interactions with both chloroquine (CI: 1.478) and artemisinin (CI: 1.440), similar to the antagonism observed between chloroquine and artemisinin (CI: 1.183). These results suggest possible interaction between this hybrid and both chloroquine and artemisinin in terms of inhibited cellular processes or intracellular targets.

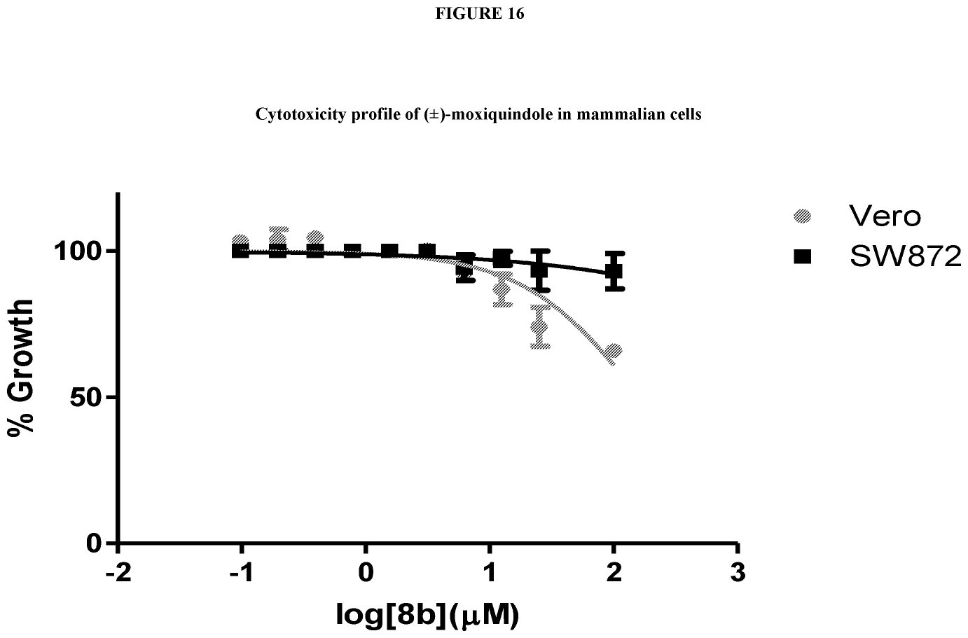

As preliminary evaluation of the compounds cytotoxicity, viability assays were conducted on monkey kidney cells (Vero) as well as using the human adipocytic cell line, SW872. As shown in FIG. 16, no significant decrease in cell viability was observed in both cell lines up to 100 μM compound concentration, indicating a selectivity index >50-fold for both mammalian cell lines. Taken together, these data suggest excellent in vitro safety of (±)-moxiquindole against these mammalian cell lines.

Hit Expansion Studies: Identification of Secondary Hits

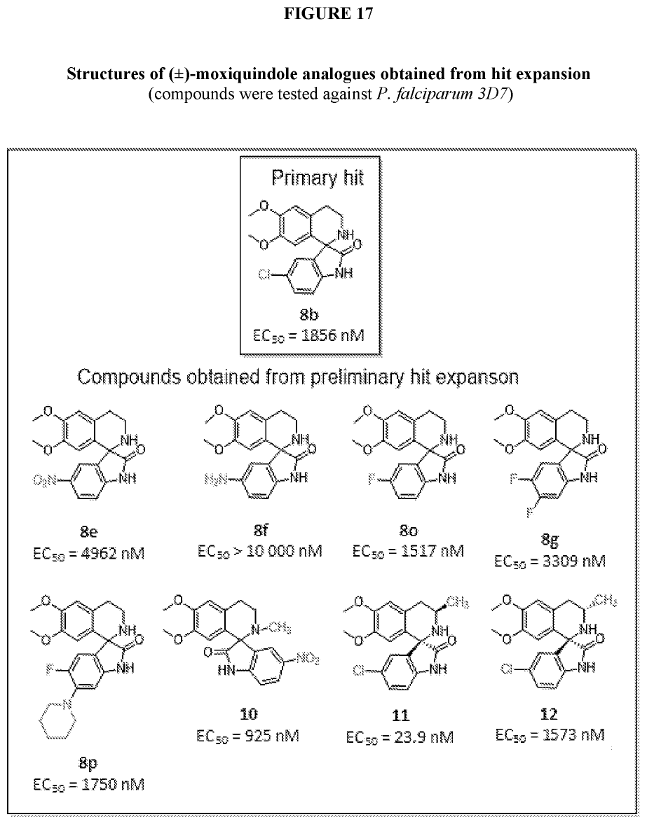

Based on the encouraging pharmacological profile of (±)-moxiquindole, hit expansion/optimization studies were initiated with a view to identifying optimized hits for preclinical lead development. The first phase of this effort led to the synthesis of eight (08) compounds, namely 8e, 6f, 6g, 8o, 8p, 10, 11 and 12, which were screened against Plasmodium falciparum CQ-sensitive 3D7 strain (FIG. 17).

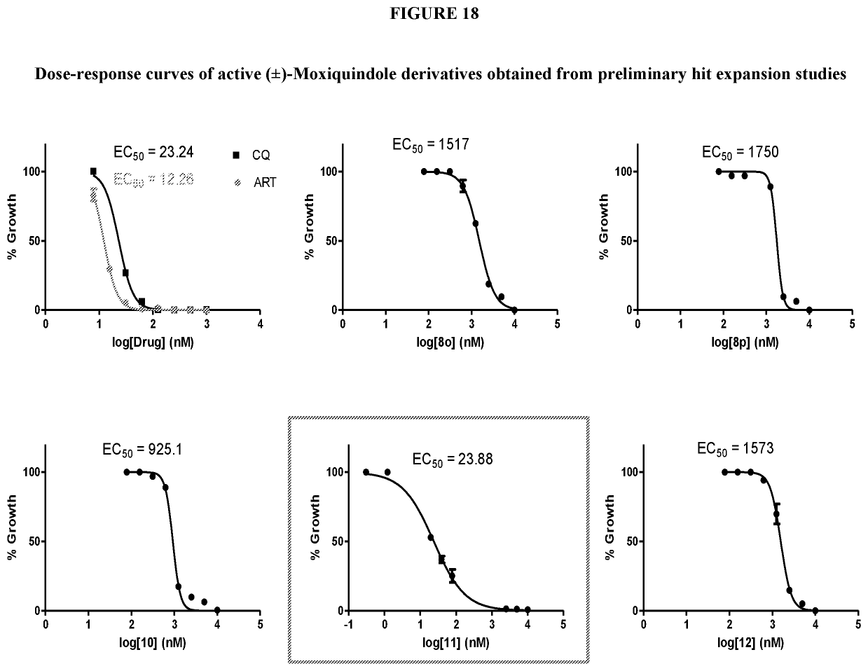

As shown in FIG. 18, five out of the eight compounds displayed activity below the cut-off point of 2 μM. One of these compounds, 11, was significantly more active than the rest, with an EC₅₀ of 24 nM (77-fold more active than the primary hit, (±)-moxiquindole), and was therefore selected for further investigation. Compound 11 has been named (±)-Homoquindole.

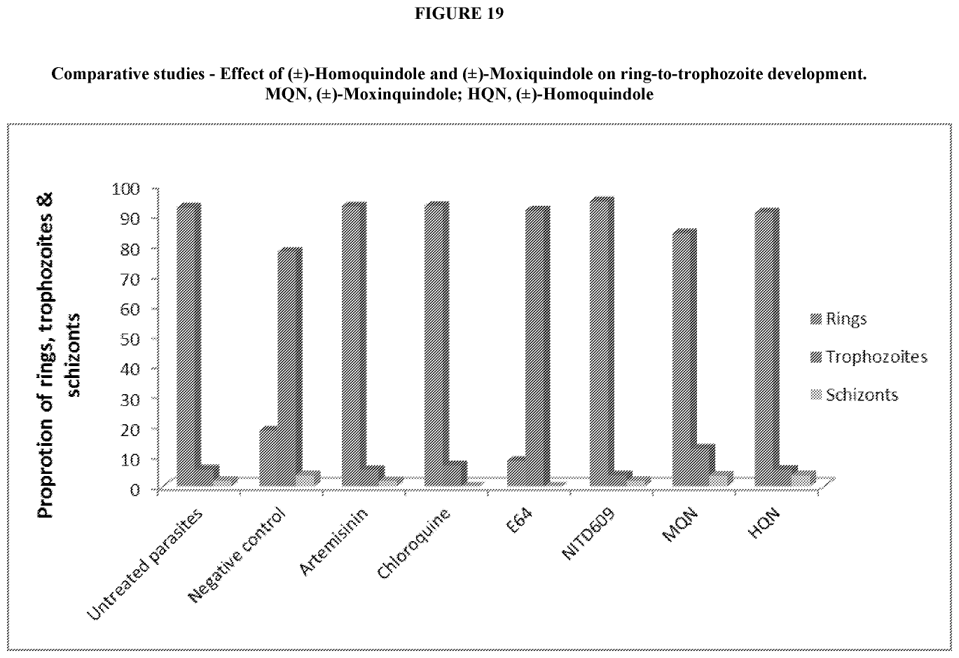

The effect of (±)-homoquindole on the asexual erythrocytic stages of P. falciparum was assessed as earlier described. (±)-Homoquindole, like (±)-moxiquindole, artemisinin (ART), chloroquine (CQ) and NITD 609, exhibits inhibitory activities against ring-to-trophozoite development, trophozoite-to-schizont development and schizont rupture as indicated by an accumulation of the respective stages in the treated parasites, relative to the solvent-treated controls (FIGS. 19-21). This indicates that spirofused tetrahydroisoquinoline-oxindole hybrids, specifically DSIIQs, constitute a novel class of multistage active antiplasmodial compounds.

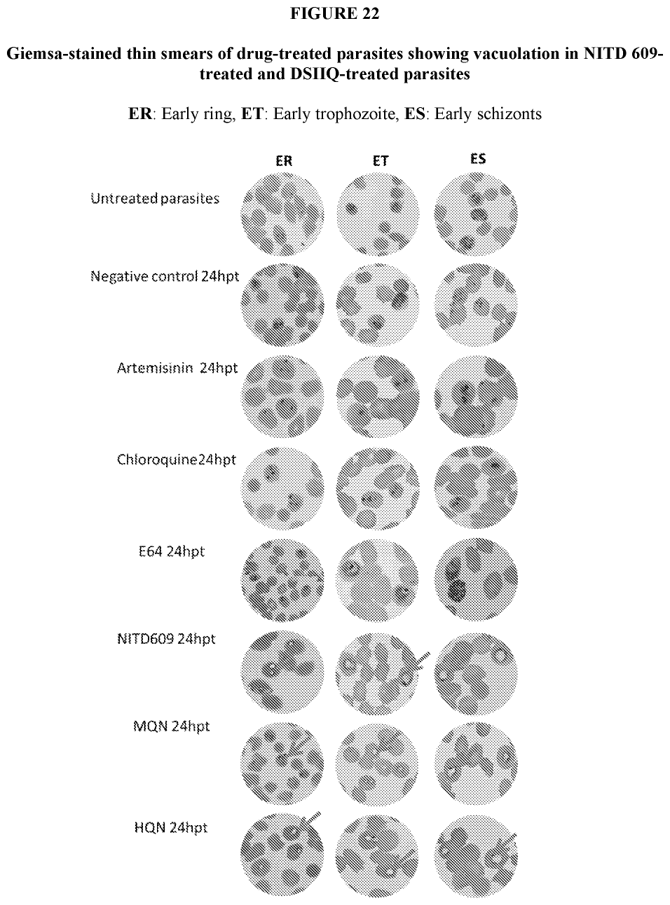

Like NITD 609, both hybrids, (±)-moxiquindole and (±)-homoquindole, induced vacuolation in parasites (FIG. 22) suggesting a similar mode of action between the spiroindolones and DSIIQs

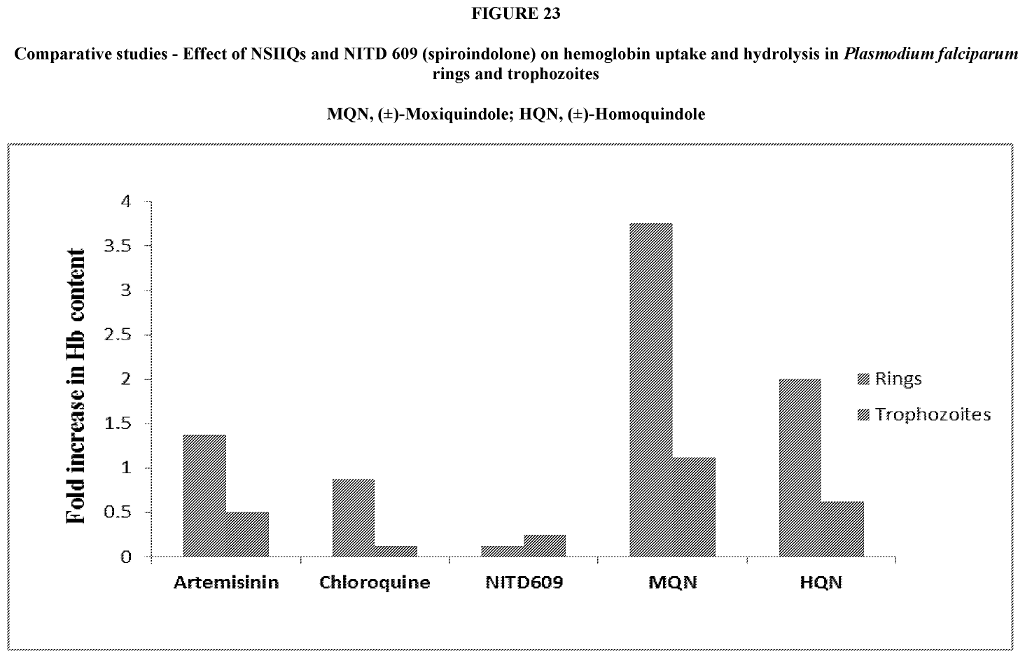

The effect of (±)-homoquindole on hemoglobin metabolism was assessed as earlier described. As shown in FIG. 23, there was an accumulation of intra-parasitic hemoglobin in both (±)-homoquindole- and (±)-moxiquindole-treated parasites, but not in the NITD609-treated, CQ-treated and ART-treated parasites. This indicates that the (±)-homoquindole is an effective accumulator of intra-parasitic hemoglobin like the (±)-moxiquindole. The data equally suggests these hybrids may act differently from NITD609 in terms of their effects on hemoglobin metabolism.

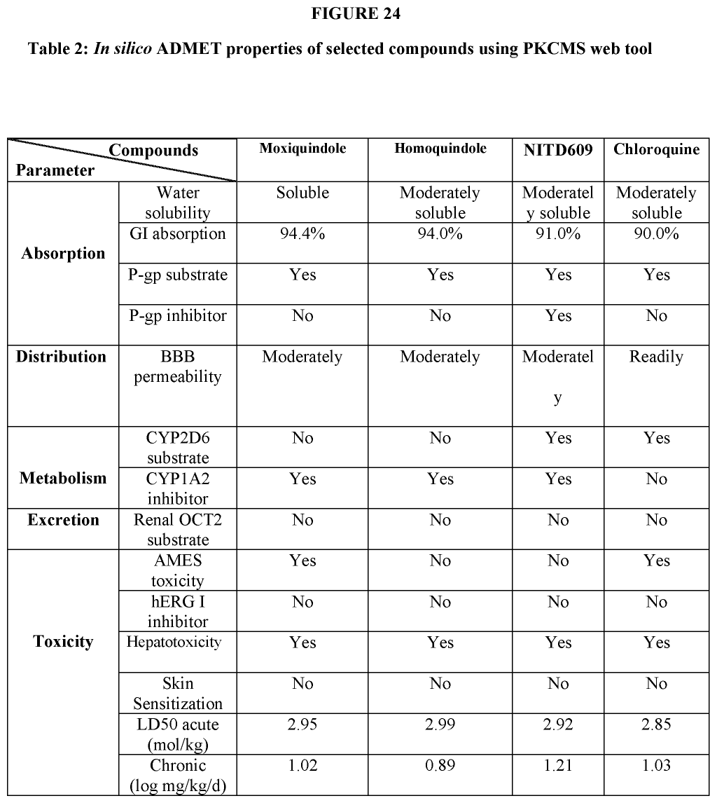

Compounds were submitted to in silico ADMET analyses to predict their overall PK properties, drug-likeness and friendliness to medicinal chemistry synthesis. Overall, both the primary and secondary hits exhibit comparable ADMET profiles with the reference oral drugs NITD609 and chloroquine (See FIG. 24—Table 2) thus may be suitable for oral or intraperitoneal administration. Furthermore, the rat oral acute toxicity score (LD50) as well as the lowest oral chronic toxicity levels of the compounds were comparable, allowing for onward efficacy testing in laboratory animals.

Drug-likeness was further predicted on the basis of Lipinski, Ghose and Veber rules and bioavailability score. The Lipinski's Rule of Five states that the absorption or permeation of a molecule is more likely when its molecular weight is under 500 g/mol, the log P value is lower than 5, and the molecule has at most 10 H-donor and H-acceptor atoms. The Ghose filter defines drug-likeness as follows: log P between −0.4 and 5.6; MW falls between 160 and 480, molar refractivity falls between 40 and 130, and the total number of atoms falls between 20 and 70.

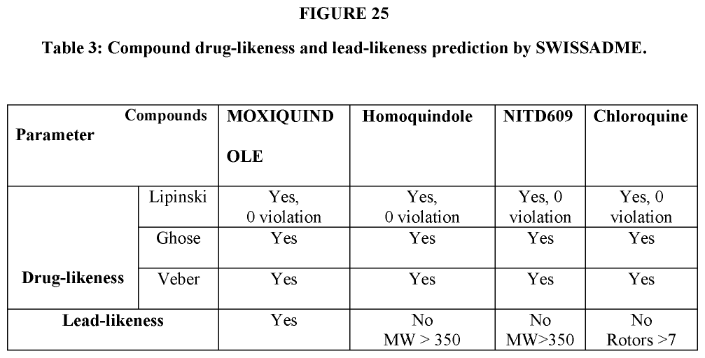

The Veber rule defines drug-likeness as rotatable bond count below 10 and polar surface area below 140. The bioavailability score indicates the probability that a compound will have at least 10% oral availability in rat or measurable Caco-2 permeability. As presented in FIG. 25, Table 3, both compounds and reference drugs meet the criteria for drug-likeness.

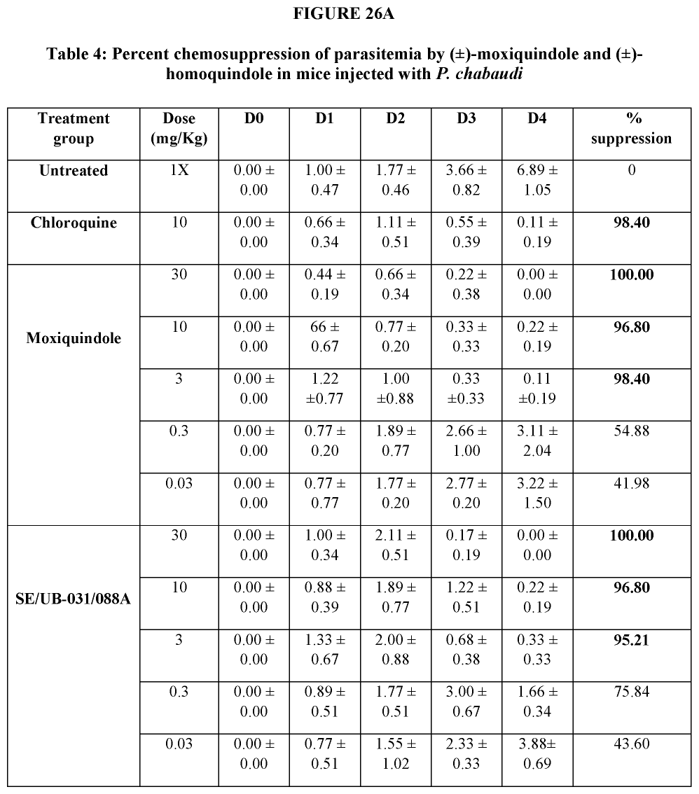

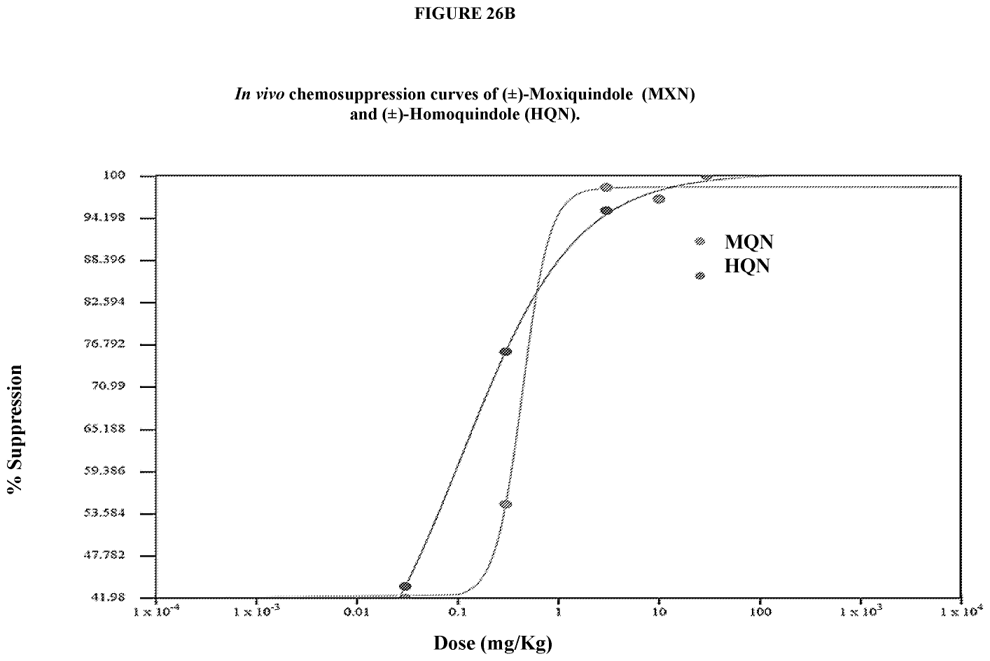

Following the encouraging in vitro antiplasmodial activities and cytotoxicity profiles reported above, as well as the ADMET properties of the primary and secondary hits identified in this study, studies were carried out to assess the in vivo efficacy of these compounds in laboratory mice. Chemosuppressive potentials were assessed using the Peter's four-day test whereas the ability of each compound to cure an established plasmodial infection was determined using the Rane's test. To assess the in vivo chemosuppressive potential of the compounds, groups of P. chabaudi-infected mice were treated by ip injection for 3 days following infection using fixed doses of 0.03, 0.3, 3, 10, or 30 mg/kg for each compound. The percent suppression of parasite growth in treated groups was calculated relative to the control group and the growth inhibition curves plotted. As shown in FIG. 26A-Table 4 & FIG. 26B, both candidate compounds (±)-moxiquindole and (±)-homoquindole displayed significant dose-dependent in vivo antimalarial activities with suppressive ED₅₀ values of 0.25 mg/Kg and 0.049 mg/Kg, respectively.

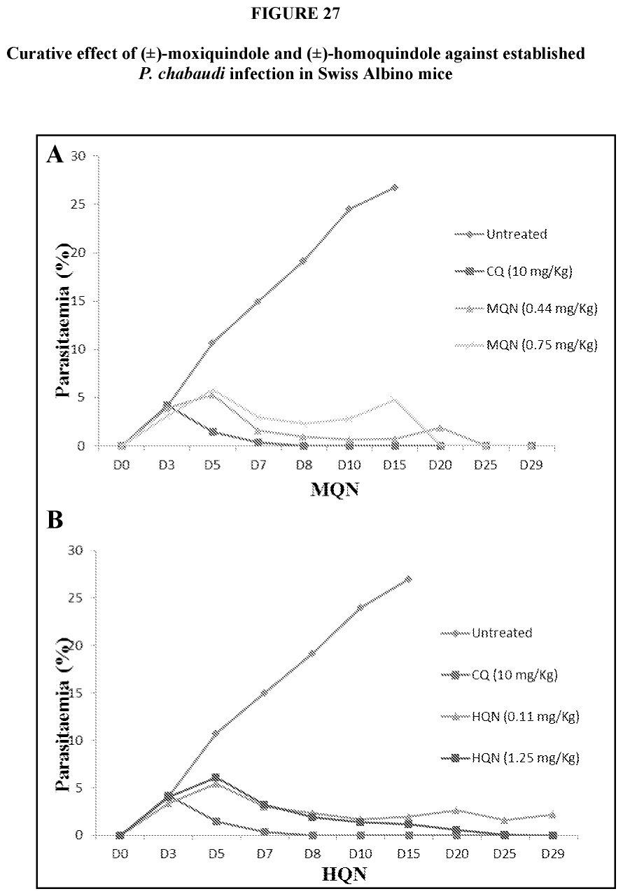

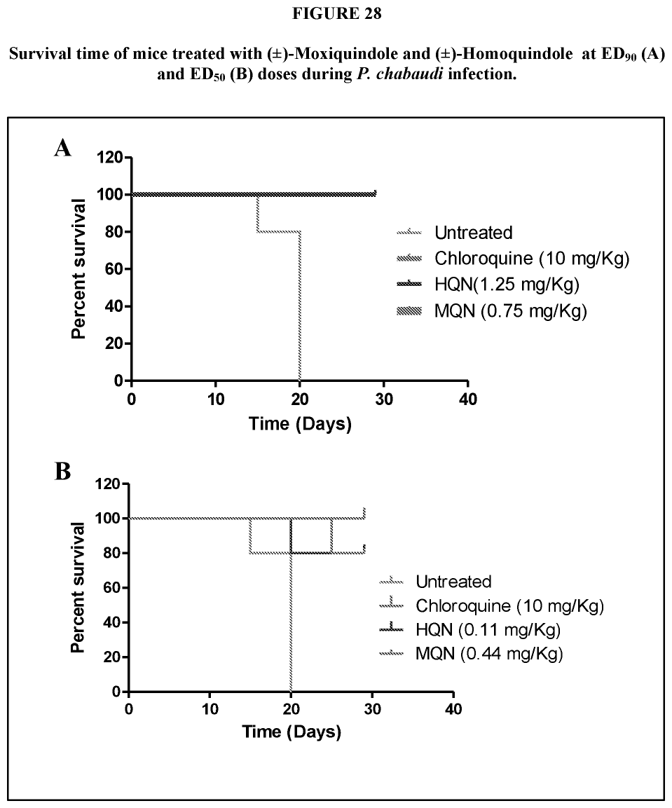

Evaluation of the curative activity of the compounds revealed that at both tested concentrations (ED₅₀ and ED₉₀); there was a progressive reduction in parasitemia by both compounds relative to the control (FIG. 27). This was observed from Day 3 and sustained for the duration of the experiment. Therefore, both compounds clearly have curative potential. Additionally, both compounds demonstrated a significant protective potential on the experimental animals as was observed in the survival time of the animals, with 100% and 80% survival at ED₉₀ and ED₅₀ concentrations, respectively (FIG. 28).

Taken together, the above data show that DSIIQs display antimalarial activity in vitro and in vivo. The representative compounds of this class, (±)-moxiquindole and (±)-homoquindole, are multi-target antimalarials that inhibit both hemoglobin metabolism (thereby depriving the parasite of aminoacids necessary for its survival) and possibly inhibit Pf Na+ ATPase, causing a catastrophic breakdown of the Na⁺ ion homeostasis in the parasite.

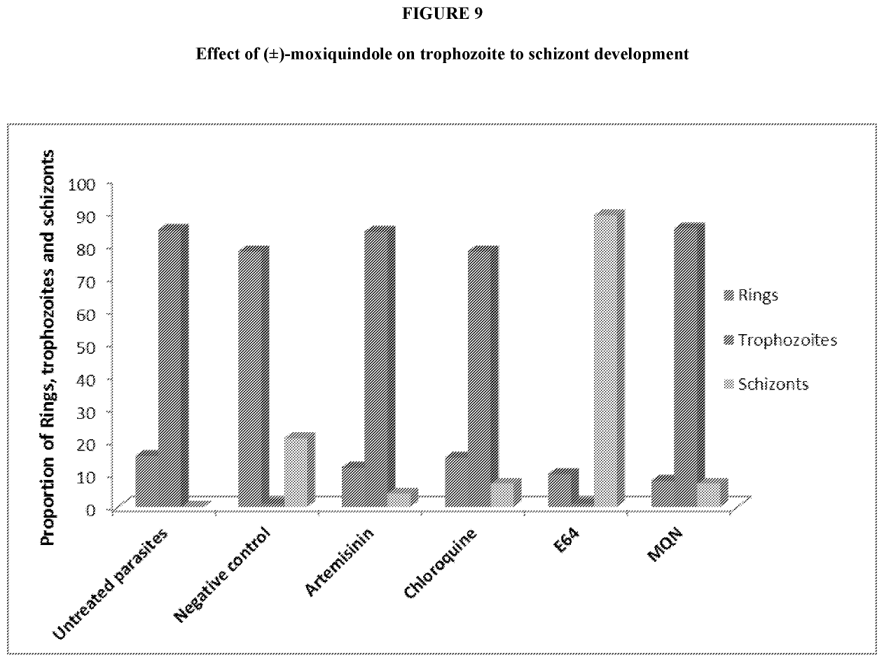

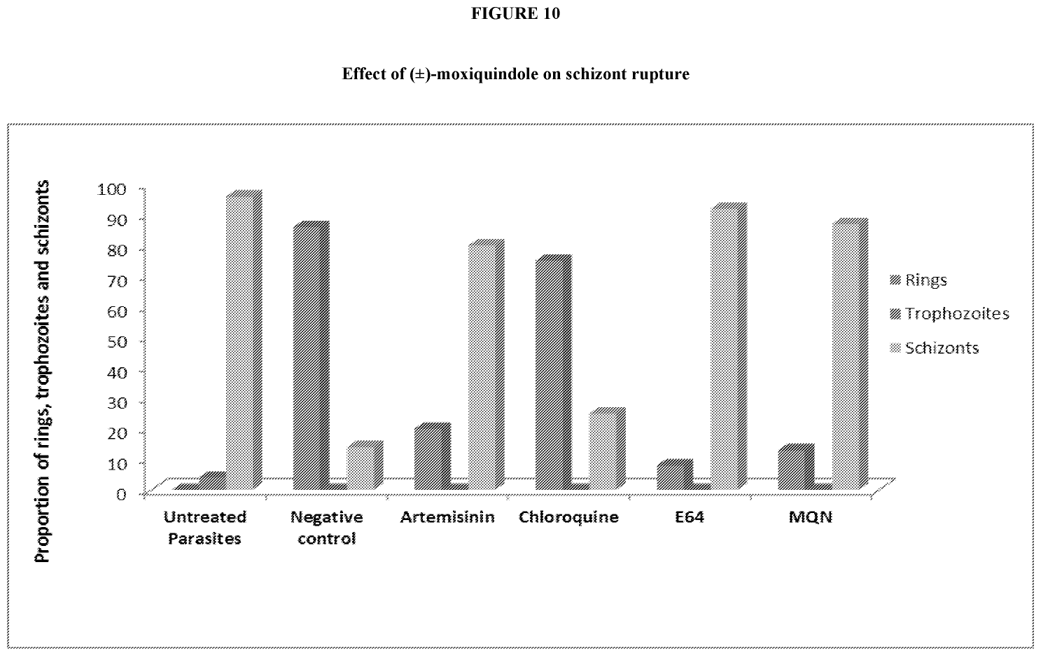

The emergence and eventual spread of resistance to artemisinin-based monotherapy led to the adoption of artemisinin-based combination therapy (ACT) as the frontline treatment strategy for malaria. Unfortunately, there are increasing and worrying reports of delayed parasite clearance following the administration of artemisinin in combination with partner drugs, suggesting a slow but inexorable emergence of resistance. To address this global problem, new antimalarial drug candidates with novel mechanisms of action have to be identified and developed. The hybridization of privileged scaffolds offers a potentially useful strategy for the discovery of compounds with novel mechanisms of action. The recently described 3′,4′-dihydro-2′H-spiro[indoline-3,1′-isoquinolin]-2-ones which are constructed from two privileged scaffolds, oxindole (OX) and 1,2,3,4-tetrahydroisoquinoline (THIQ), can be considered as hybrids of two powerful but mechanistically dissimilar classes of antimalarials: spiroindolones and naphthylisoquinolines. The combination of these two properties into one molecule would be expected to produce a potent and highly efficacious antimalarial agent. In the current study, the screening of a small library of DSIIQs against the CQ-sensitive Pf 3D7 strain resulted in the identification of a hit, named (±)-moxiquindole. This compound displayed potent inhibitory activity against all strains examined (EC50 Pf 3D7=1.85 μM; EC50 for Pf Dd2=1.73 μM), suggesting no cross-resistance with known antimalarials including pyrimethamine and chloroquine, and prompting further investigation of this compound as a representative of this class of hybrids. As a complementary screen, the cytotoxicity of this compound on Normal African Green Monkey Kidney Epithelial (Vero) and the Human Liposarcoma (SW872) cell lines was evaluated using an MTT cell proliferation assay. The CC50 value of (±)-moxiquindole against both cell lines was >100 μM resulting in over fifty fold selectivity (FIG. 16). Subsequently, the developmental stage specific action of (±)-moxiquindole was studied by microscopy. Following the invasion of erythrocytes, malaria parasite merozoites attain maturity by passing through a series of developmental stages termed ring, trophozoite, schizont, and segmenter. Antimalarials exert their actions by interfering with one or more stages in the life cycle of the parasite; therefore, knowledge of the stage specific action of a test compound can provide useful insights into the mode of action. In the current investigation, the mode of action of (±)-moxiquindole was compared with two antimalarials in use (artemisinin and chloroquine) and E64 (a cysteine protease inhibitor), by determining their inhibitory effects on the intraerythrocytic stages of the parasite, schizont rupture and merozoite invasion. In a previous study of all antimalarials then in use, only artemisinin displayed significant inhibition of schizont maturation but no compound inhibited merozoite invasion (Thomas et al., 2018). Consistent with these reports, the present study found that artemisinin has a significant inhibitory effect on schizont maturation and eventual merozoite egress, as the schizonts appeared dead compared to the viable rings in the untreated controls. Chloroquine on the other hand, had no effect on schizont rupture as revealed by the presence of rings 24 hours following drug treatment although it had a significant effect on schizont development as seen in FIG. 10. The test compound, rapidly arrested parasite development at the early ring (FIG. 8), mid trophozoite (FIG. 9), and schizont stages (FIG. 10). The presence of schizonts following a 24-hour treatment with (±)-moxiquindole contrasts sharply with the presence of rings in the untreated controls and is indicative of an arrest in schizont rupture. Merozoite egress, which is preceded by schizont rupture, is a protease-dependent sequence of membrane permeabilization and eventual breakage (Hale et al., 2017; Wilson et al., 2013); therefore it is reasonable to attribute the blockade of schizont rupture by (±)-moxiquindole to protease inhibition. Consistent with this suggestion, the cysteine protease inhibitor E64 was also found to arrest merozoite egress in the present study (FIG. 10) as expected from previous reports (Lee et al., 2014; Lehmann et al., 2018). Proteases have been implicated in a wide range of malaria parasite metabolic processes including hemoglobin degradation; therefore, additional investigations were carried out in order to gain a better understanding of the actions of the test compound. Indeed, the antimalarial activity of quinoline-based antimalarials is attributed to their interference with hemoglobin metabolism either by inhibiting the uptake of hemoglobin by the parasite, or by inhibiting degradation of hemoglobin, or by binding the toxic hematin and preventing hemozoin formation, all of which result in the eventual death of the parasite (Herraiz et al., 2019; Hoppe et al., 2004; Prasad et al., 2013). In the present study, both ring and trophozoite stage parasites treated with (±)-moxiquindole were found to accumulate hemoglobin to a greater extent than parasites treated with either chloroquine or artemisinin (FIG. 12). Although some hemoglobin degradation is observed during schizont development, the vast majority of degradation occurs during trophozoite development. As expected, hemoglobin accumulation was higher in drug-treated ring stage parasites compared to drug-treated trophozoite stage parasites (FIG. 12). Previous reports have showed that hemoglobin degradation is an ordered process initiated by aspartic-protease cleavages, followed by cysteine protease action. As such, the effect of cysteine protease inhibitors is minimal compared to the effect of aspartic protease inhibitors as far as hemoglobin degradation is concerned (Francois et al., 1997). Consistent with these reports, E64, a known cysteine protease inhibitor demonstrated a minimal effect on hemoglobin degradation compared to the test compound. These observations further support the view that (±)-moxiquindole is indeed acting as a protease inhibitor and a more effective accumulator of intra-parasitic hemoglobin; thus, depriving the organism of an essential source of substrates for energy metabolism.

Further support for this proposed mode of action is provided by combination studies which paired (±)-moxiquindole with either chloroquine or artemisinin. As presented above, the interaction of the test compound with either antimalarial was antagonistic (FIGS. 14 & 15). The actions of both chloroquine and artemisinin depend on the presence of heme, a metabolite generated by protease catalyzed degradation of hemoglobin. Drug induced blockade of the latter process, with resulting depletion of heme levels, would therefore be expected to negatively affect the actions of these two compounds as observed in the present study. Similar observations have been reported in a study of the interaction between chloroquine and both aspartic and cysteine proteases using the same analytic methods (Mungthin et al., 1998).

Previous studies have shown that during intraerythrocytic development, the phospholipid content of the parasite increases significantly as the latter generates membranes needed for growth and division (Wijayanti et al., 2010). Though some of the lipids are synthesized in the apicoplast, the bulk of the lipids are scavenged from the host. These lipids are metabolized to generate neutral lipid bodies which nucleate the formation of hemozoin in the parasite digestive (food) vacuole (Jackson et al., 2004). Consequently, the lipid metabolic pathway is an important target in antimalarial drug discovery. In the continuing investigation of the mode of action of (±)-moxiquindole, the actions of this compound on lipid metabolism were compared with those of chloroquine, artemisinin and E64. Trophozoite stage parasites treated with either, artemisinin or E64 clearly revealed the presence of stained neutral lipid bodies indicating that these compounds have no effect on vacuolar lipid uptake. On the other hand, parasites treated with either chloroquine or the test compound were devoid of neutral lipid bodies clearly suggesting interference with parasite vacuolar lipid dynamics (FIG. 13). This observations support previous findings that chloroquine and other quinoline-based compounds bind toxic ferriprotoporphyrin IX and inhibit dimerization (Fitch et al., 2003), and further point to an additional target for the DSIIQs. Based on the totality of the evidence, (±)-moxiquindole therefore emerged as a rapidly acting multi-target antimalarial that combines the modes of action of the spiroindolones and the NIQs.

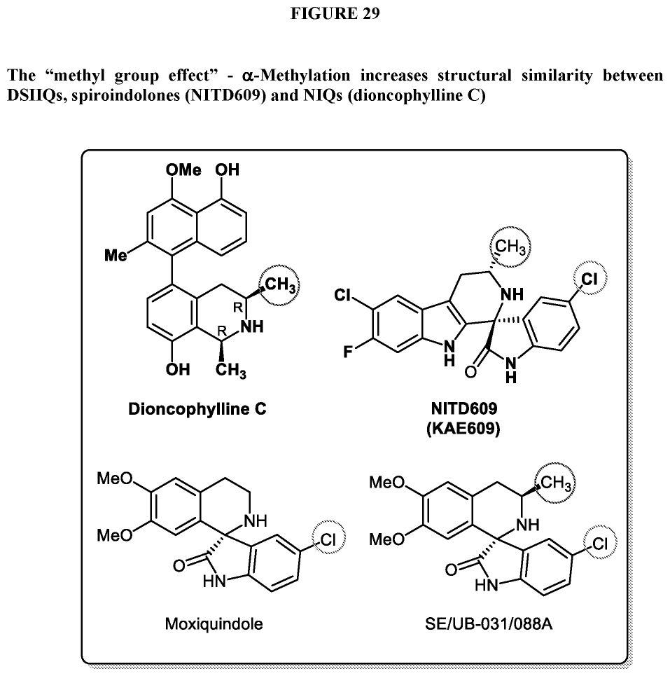

Based on these findings, hit expansion studies were launched to identify more active compounds for subsequent investigation and lead identification. The first stage of this effort resulted in the identification of five active compounds, including (±)-homoquindole that is 80 times more potent than (±)-moxiquindole. Due to the large increase in potency, the structure of (±)-homoquindole was carefully analyzed for possible clues to the origins of this enhancement of antiplasmodial activity. A brief inspection reveals that (±)-moxiquindole and (±)-homoquindole differ only by a single methyl group found within the piperidyl sub-fragment of the THIQ scaffold. Remarkably, a similar methyl group is found at the corresponding position in both the NIQs and the spiroindolones, represented here by dioncophylline C and NITD609, respectively (FIG. 29). In the spiroindolone series, removal of this methyl group results in a drastic reduction of antiplasmodial activity (Turner, 2016). Therefore, enhanced structural similarity between the DSIIQs, NIQs and spiroindolones leads to increased antiplasmodial activity. In other words, as the hybrid becomes more like the constituent elements, the better it performs. Since the methyl group appears to be critical for activity in both the spiroindolones and DSIIQ series, it is reasonable to conclude that both classes of compounds act by a similar mode of action.

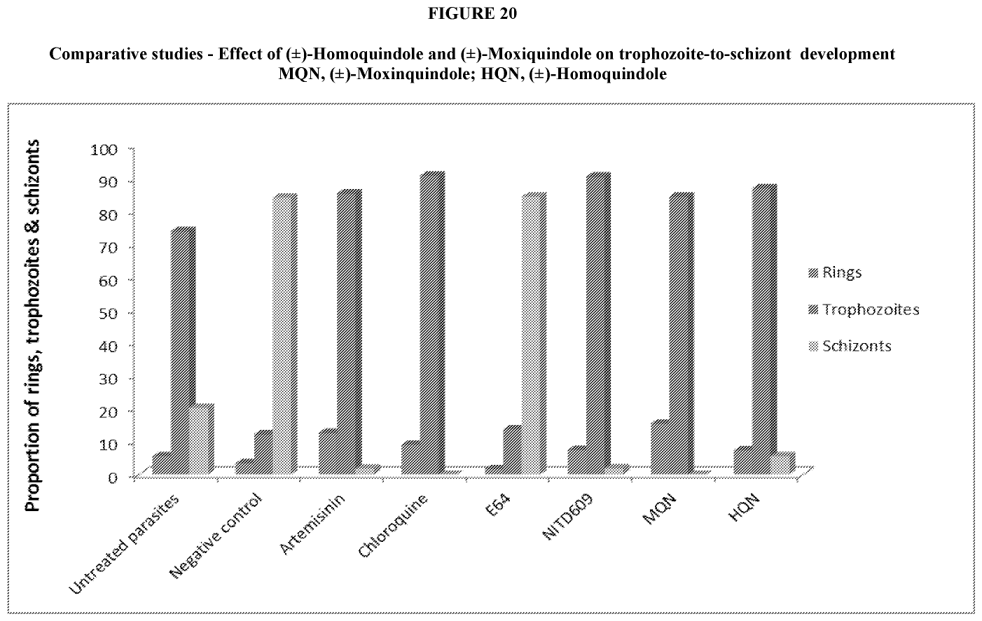

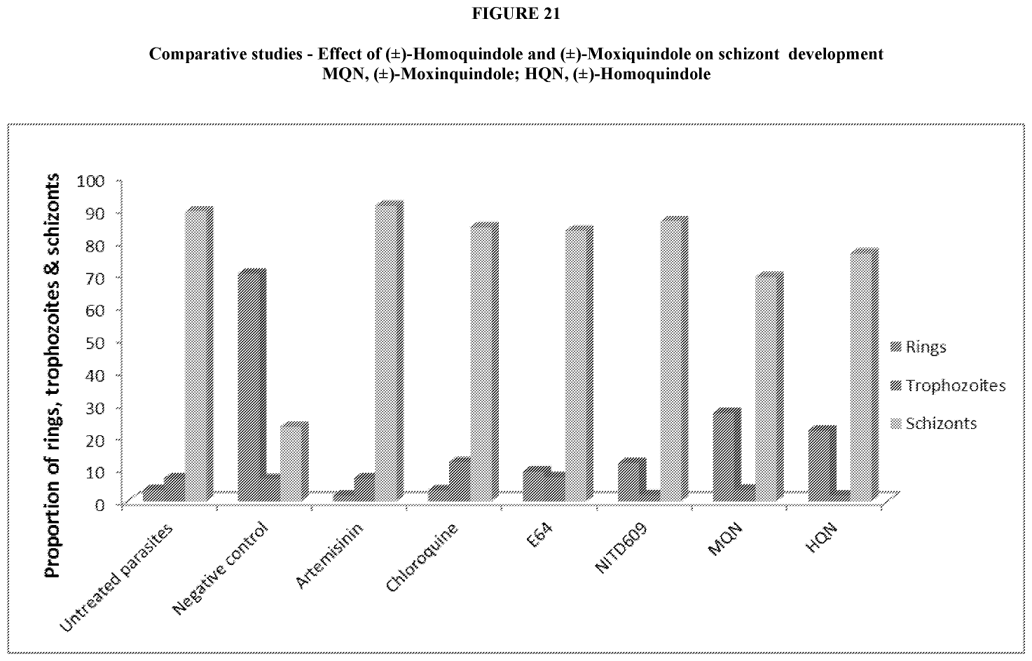

Mode-of-action studies of the secondary hit (±)-homoquindole reveal striking similarities between this compound and the primary hit (±)-moxiquindole in all aspects examined. Accordingly, both compounds were found to inhibit all stages of the erythrocytic cycle: the development of rings to trophozoites (FIG. 19), trophozoites to schizonts (FIG. 20) and schizont development (FIG. 21). Similar multistage inhibition of the erythrocytic cycle was observed for NITD609 (FIGS. 19-21), consistent with previous findings (Rottmann et al., 2010; Turner, 2016) and has also been reported for the NIQs (Moyo et al., 2020). The DSIIQs therefore parallel both the NIQs and spiroindolones in this aspect. Additionally, both (±)-homoquindole and (±)-moxiquindole were found to inhibit hemoglobin degradation, a property associated with the THIQ pharmacophore and shared with the NIQs but not NITD609. We therefore conclude that the DSIIQs constitute a novel class of multi-target antimalarials that display multistage inhibitory activity on the erythrocytic cycle of P. falciparum. These compounds act by combining the modes of action of two classes of antimalarial agents: inhibition of hemoglobin metabolism, a property of the NIQs, and possibly inhibition of Pf Na+-ATPase, a property of the spiroindolones. The 3′,4′-dihydro-2′H-spiro[indoline-3,1′-isoquinolin]-2-one scaffold is therefore a true molecular hybrid of the spiroindolones and NIQs.

As a prelude to in vivo studies, the primary and secondary hits were subjected to pharmacokinetic evaluation in silico. Both compounds showed suitable pharmacokinetic profiles based on accepted parameters, and were thus submitted for evaluation in mice. In the chemosuppression assay, (±)-homoquindole was found to show a slight advantage over (±)-moxiquindole at 0.3 mg/kg; however, both compounds were equally effective at 3.0 mg/kg, providing 98% suppression of parasitemia. This was unexpected, given the large disparity in potency between these compounds in vitro but it may be attributed to differences in the disposition of the two compounds which are not apparent from the in silico pharmacokinetics studies. ED50 values in vivo showed only a five-fold difference compared with an 80-fold difference in EC50 values in vitro (vide supra). Chloroquine provided a comparable level of suppression of parasitemia at a dose of 10 mg/kg. In the subsequent curative study, both compounds displayed a comparable curative potential at the ED50 and ED90 values. At the ED90, all treated animals survived the end of the 28-day test period while most of the untreated animals died. The 3′,4′-dihydro-2′H-spiro[indoline-3,1′-isoquinolin]-2-ones (DSIIQs) have therefore shown promise as multi-target antimalarials that deserve further investigation.

### Experimental

Chemistry

The synthesis of the target compounds has been described (Lobe & Efange, 2020). Previously unreported compounds are described herein. The compounds were provided in racemic form and tested as the hydrochlorides.

### Synthesis of 6′,7′-dimethoxy-5-nitro-3′,4′-dihydro-2′H-spiro[indoline-3,1′-isoquinolin]-2-one (8e)

Prepared from 5-nitroisatin (2.0 g, 10.4 mmol), 3,4-dimethoxyphenethylamine (2.3 g 12.5 mmol) and polyphosphoric acid (5 g), as described earlier (see Method G in Lobe & Efange, 2020). The crude product was purified by flash chromatography (hexane:ethyl acetate—70:30). Yield, 2.9 g, 78% (brown solid), M.p. 174-175° C.

¹H NMR (DMSO-d₆, 600 MHz): δ ppm 2.69 (dt, J=15.9, 3.8 Hz, 1H, H4′a), 2.92 (ddd, J=15.5, 9.8, 5.5 Hz, 1H, H4′b), 2.97-3.04 (m, 1H, H3′a), 3.59.3.63 (m, 1H, H3′b), 3.34 (s, 3H, 7′-OCH₃), 3.75 (s, 3H, 6′-OCH₃), 5.88 (s, 1H, H8′), 6.79 (s, 1H, H5′), 7.11 (d, J=8.7 Hz, 1H, H7), 7.81 (d, J=2.4 Hz, 1H, H4), 8.22 (dd, J=8.7, 2.4 Hz, 1H, H6), 10.99 (s, 1H, H1). ¹³C NMR (DMSO-d₆, 150 MHz): δ ppm 28.5 (C4′), 38.5 (C3′), 55.9 (7′-OCH₃), 56.1 (6′-OCH₃), 63.6 (C3/C1′), 109.4 (C8′), 110.4 (C7), 113.1 (C5′), 120.5 (C4), 125.6 (C8′a), 126.7 (C6), 129.8 (C4′a), 137.0 (C3a), 142.8 (C7a), 147.6 (C7′), 148.8 (C6′), 149.4 (C5), 180.8 (C2). FTMS+cESI: m/z 356.12 [M+1]⁺.

### Synthesis of 5-amino-6′,7′-dimethoxy-3′,4′-dihydro-2′H-spiro[indoline-3,1′-isoquinolin]-2-one (8f)

Compound 8f was obtained from the reduction of the 5-nitro group of previously synthesized 8e (1.0 g, 2.2.8 mmol) and zinc dust (0.64 g 9.8 mmol, 3.5 eq). To a warm ethanolic solution of 8e were added portions of zinc dust and concentrated HCl over 2-minute intervals. Upon complete addition of the reagents, the reaction mixture was refluxed for 1 hour. During this time there was complete consumption of 8e as observed on TLC. Reaction mixture was concentrated under reduced pressure, made basic to pH 9 by the addition of saturated aqueous sodium bicarbonate. Within the process, insoluble zinc carbonate was precipitated out and filtered off by suction filtration. Product was extracted into ethyl acetate (30 mL×2), combined organic extracts dried over anhydrous sodium sulphate and concentrated under reduced pressure. The crude product was purified by flash chromatography (hexane:ethyl acetate—20:80). Yield, 0.8 g, 89% (brown solid). M.p. 186-187° C.

¹H NMR (DMSO-d₆, 700 MHz): δ ppm 2.63-2.70 (m, 1H, H4′a), 2.80 (ddd, J=15.9, 8.6, 5.3 Hz, 1H, H4′b), 2.98 (dt, J=12.4, 5.1 Hz, 1H, H3′a), 3.57 (ddd, J=12.7, 8.7, 4.4 Hz, 1H, H3′b), 3.42 (s, 3H, 7′-OCH₃), 3.73 (s, 3H, 6′-OCH₃), 5.92 (s, 1H, H8′), 6.34 (d, J=2.3 Hz, 1H, H4), 6.42 (dd, J=8.2, 2.3 Hz, 1H, H6), 6.59 (d, J=8.2 Hz, 1H, H7), 6.71 (s, 1H, H5′), 9.87 (s, 1H, H1). ¹³C NMR (DMSO-d₆, 175 MHz): δ ppm 28.8 (C4′), 38.8 (C3′), 55.9 (7′-OCH₃), 56.1 (6′-OCH₃), 64.1 (C3/C1′), 110.0 (C8′), 110.41 (C7), 112.0 (C4), 112.8 (C5′), 113.8 (C6), 128.0 (C8′a), 129.3 (C4′a), 132.0 (C7a), 137.2 (C3a), 144.4 (C5), 147.4 (C7′), 148.4 (C6′), 180.2 (C2). FTMS+cESI: m/z 324.13 [M−1]⁺.

### Synthesis of 5,6-difluoro-6′,7′-dimethoxy-3′,4′-dihydro-2′H-spiro[indoline-3,1′-isoquinolin]-2-one (8g)

Prepared from 5,6-difluoroisatin (1.0 g, 5.5 mmol, 1 eq), 3,4-dimethoxyphenethylamine (1.2 g, 6.6 mmol) and polyphosphoric acid (5 g), following published methods (see Method G in Lobe & Efange, 2020). The crude product was purified by flash chromatography (hexane:ethyl acetate—70:30). Yield, 1.3 g, 68% (white solid), M.p. 220-221° C.

¹H NMR (DMSO-d₆, 700 MHz): δ ppm 2.69 (dt, J=15.9, 4.1 Hz, 1H, H4′a), 2.84 (ddd, J=15.2, 9.3, 5.4 Hz, 1H, H4′b), 2.98 (dt, J=11.4, 4.7 Hz, 1H, H3′a), 3.45 (s, 3H, 7′-OCH₃), 3.57-3.64 (m, 1H, H3′b), 3.74 (s, 3H, 6′-OCH₃), 5.88 (s, 1H, H5′), 6.75 (s, 1H, H8′), 6.93 (dd, J=10.5, 6.6 Hz, 1H, H7), 7.09 (dd, J=9.8, 7.9 Hz, 1H, H4), 10.40 (s, 1H, H1). ¹³C NMR (DMSO-d₆, 175 MHz): δ ppm 28.5 (C4′), 38.5 (C3′), 55.9 (7′-OCH₃), 56.1 (6′-OCH₃), 63.8 (C3/C1′), 99.8 (C7), 109.5 (C5′), 113.0 (C8′), 114.6 (C4), 126.3 (C8′a), 129.6 (C4′a), 132.1 (C3a), 139.3 (C7a), 147.6 (C7′), 148.8 (C6′), 145.2 (C5), 149.4 (C6), 180.5 (C2). FTMS+cESI: m/z 347.12 [M+1]⁺.

### Synthesis of 5-fluoro-6′,7′-dimethoxy-3′,4′-dihydro-2′H-spiro[indoline-3,1′-isoquinolin]-2-one (8o)

Prepared from 5-fluoroisatin (1.0 g, 6.1 mmol, 1 eq), 3,4-dimethoxyphenethylamine (1.3 g, 7.3 mmol, 1.2 eq) and polyphosphoric acid (2 g), as previously described (see Method G in Lobe & Efange, 2020). The crude product was purified by flash chromatography (hexane:ethyl acetate—70:30). Yield, 1.1 g, 55% (brown solid), M.p. 98-99° C.

¹H NMR (DMSO-d₆, 600 MHz): δ ppm 2.67 (dt, J=15.9, 4.2 Hz, 1H, H4′a), 2.84 (ddd, J=15.2, 9.1, 5.3 Hz, 1H, H4′b), 2.99 (ddd, J=12.5, 5.4, 4.1 Hz, 1H, H3′a), 3.57-3.63 (m, 1H, H3′b), 3.43 (s, 3H, 7′-OCH₃), 3.74 (s, 3H, 6′-OCH₃), 5.887 (s, 1H, H8′), 6.75 (s, 1H, H5′), 6.85-6.91 (m, 2H, H4, H7), 7.06 (dd, J=9.6, 8.5, 2.7 Hz, 1H, H6), 10.31 (s, 1H, H1). ¹³C NMR (DMSO-d₆, 150 MHz): δ ppm 28.6 (C4′), 38.6 (C3′), 55.9 (7′-OCH₃), 56.1 (6′-OCH₃), 64.2 (C3/C1′), 109.5 (C8′), 110.8 (C7), 112.6 (C5′), 113.0 (C4), 115.5 (C6), 126.6 (C8′a), 129.6 (C4′a), 138.8 (C7a), 147.5 (C7′), 148.6 (C6′), 157.8 (C3a), 159.3 (C5), 180.5 (C2). FTMS+cESI: m/z 329.13 [M+1]⁺.

### Synthesis of 5-fluoro-6′,7′-dimethoxy-6-(piperidin-1-yl)-3′,4′-dihydro-2′H-spiro[indoline-3,1′-isoquinolin]-2-one (8p)

Prepared from previously synthesized 5-fluoro-6-(piperidin-1-yl)indoline-2,3-dione (1.4 g, 5.6 mmol), 3,4-dimethoxyphenethylamine (1.23 g, 6.8 mmol) and polyphosphoric acid (3 g) (Method G, Lobe & Efange, 2020). The crude product was purified by column chromatography (hexane:ethyl acetate—40:60).

Yield, 1.32 g, 57% (yellow oil). ¹H NMR (DMSO-d₆, 600 MHz): δ ppm 1.53 (m, 2H, piperidin-1-yl), 1.67-1.64 (m, 4H, piperidin-1-yl), 2.652.65 (m, 1H, H4′a), 2.84 (m, 1H, H4′b), 2.99-2.94 (m, 5H, Piperidin-1-yl, H3′a), 3.45 (s, 3H, 7′-OCH₃), 3.56 (m, 1H, H3′b), 3.74 (s, 3H, 6′-OCH₃), 5.90 (s, 1H, H5′), 6.74 (s, 1H, H8′), 6.79 (s, 1H, H4), 6.81 (s, 1H, H7). ¹³C NMR (DMSO-d₆, 150 MHz): δ ppm 21.2 (piperidin-1-yl), 24.2 (piperidin-1-yl), 35.1 (C4′), 39.3 (C3′), 51.9 (piperidin-1-yl), 55.9 (7′-OCH₃), 56.1 (6′-OCH₃), 63.9 (C3/C1′), 101.3 (C7), 109.7 (C5′), 112.4 (C8′), 112.9 (C4), 120.9 (C8′a), 121.2 (C4′a), 132.1 (C3a), 139.0 (C7a), 147.5 (C7′), 147.7 (C6′), 149.1 (C6), 150.4 (C5), 172.5 (C2), FTMS+cESI: m/z 412.20 [M+1]⁺.

### Synthesis of 6′,7′-dimethoxy-2′-methyl-5-nitro-3′,4′-dihydro-2′H-spiro[indoline-3,1′-isoquinolin]-2-one (10)

Prepared from 6′,7′-dimethoxy-5-nitro-3′,4′-dihydro-2′H-spiro[indoline-3,1′-isoquinolin]-2-one (8e). (1 g, 2.8 mmol) and formaldehyde (0.5 mL of 37% formalin, 4.2 mmol, 1.5 eq). The crude product was purified by flash chromatography (hexane:ethyl acetate—50:50). Yield, 0.6 g, 58%. (brown solid). M.p. 137-138° C.

¹H NMR (DMSO-d₆, 700 MHz): δ ppm 2.10 (s, 3H, N2′-CH₃), 2.80 (dt, J=16.7, 3.7 Hz, 1H, H4′a), 2.91 (m, 1H, H3′a), 3.02-3.10 (m, 1H, H4′b), 3.39 (s, 3H, 7′-OCH₃), 3.49-3.543.63 (m, 1H, H3′b), 3.74 (s, 3H, 6′-OCH₃), 5.84 (s, 1H, H8′), 6.81 (s, 1H, H5′), 7.14 (d, J=8.7 Hz, 1H, H7), 7.70 (d, J=2.4 Hz, 1H, H4), 8.25 (dd, J=8.8, 2.4 Hz, 1H, H6), 11.12 (s, 1H, H1). ¹³C NMR (DMSO-d₆, 175 MHz): δ ppm 28.6 (C4′), 39.6 (N2′-CH₃), 46.94 (C3′), 55.9 (7′-OCH₃), 56.1 (6′-OCH₃), 69.2 (C3/C1′), 109.6 (C7), 110.6 (C8′), 112.5 (C5′), 120.3 (C4), 125.3 (C8′a), 126.6 (C6), 128.5 (C4′a), 135.0 (C3a), 143.2 (C7a), 147.7 (C7′), 148.8 (C6′), 149.6 (C5), 177.3 (C2). FTMS+cESI: m/z 370.14 [M+1]⁺.

### Synthesis of 5-chloro-6′,7′-dimethoxy-3′-methyl-3′,4′-dihydro-2′H-spiro[indoline-3,1′-isoquinolin]-2-one (11 and 12)

α-Methyl-3,4-dimethoxyphenethylamine was prepared from 3,4-dimethoxybenzaldehyde and nitroethane as described for the α-methyltryptamines in Yeung et al. (2010). The target compounds were subsequently prepared from 5-chloroisatin (1 g, 5.5 mmol), α-methyl-3,4-dimethoxyphenethylamine (1.3 g, 6.6 mmol) and polyphosphoric acid (2 g) as described in Method G (Lobe & Efange, 2020) The reaction afforded two diasteromeric racemates that were separated by column chromatography (hexane:ethyl acetate—60:40). The more mobile diastereomer was assigned the trans-structure 11, while the less mobile isomer was assigned the cis structure 12, The assignment of structure is based on the chemical shifts of C3′ and H3′ in the C-13 and ¹H spectra, respectively, coupled with comparison with the spectra of the spiroindolone series, which show the signal of the methine proton at C3′ of NITD609 as deshielded relative to that of the other diastereomer) (Zou et al., 2012).

Compound 11: Yield, 0.3 g, 15% (white solid). ¹H NMR (DMSO-d₆, 400 MHz): δ ppm 1.07 (d, J=6.3 Hz, 3H, —N2′C3′HCH₃), 2.46-2.44 (m, 1H, H4′a), 2.66 (dd, J=15.7 Hz, 1H, H4′b), 3.41 (s, 3H, 7′-OCH₃), 3.72 (s, 3H, 6′-OCH₃), 3.95-3.83 (m, 1H, H3′), 5.85 (s, 1H, H8′), 6.71 (s, 1H, H5′), 6.88 (d, J=8.2 Hz, 1H, H7), 6.96 (d, J=2.2 Hz, 1H, H4), 7.26 (dd, J=8.3, 2.2 Hz, 1H, H6), 10.29 (s, 1H, H1).

¹³C NMR (DMSO-d₆, 100 MHz): δ ppm 22.2 (—CCH3), 36.9 (C4′), 43.4 (C3′), 55.9 (7′-OCH₃), 56.2 (6′-OCH₃), 64.9 (C3/C1′), 109.5 (C8′), 111.3 (C7), 112.8 (C5′), 121.2 (C3a), 125.1 (C4), 126.2 (C8′a), 129.0 (C6), 129.9 (C4′a), 137.8 (C5), 141.8 (C7a), 147.5 (C7′), 148.6 (C6′), 172.4 (C2), FTMS+cESI: m/z 359.29 [M+1]⁺.

Compound 12: Yield, 0.54 g, 27% (white solid). ¹H NMR (DMSO-d₆, 400 MHz): δ ppm 1.12 (d, J=6.2 Hz, 3H, —N2′C3′HCH₃), 2.48-2.39 (m, 1H, H4′a), 2.77 (dd, J=16.0 Hz, 1H, H4′b), 3.38 (m, 1H, H3′), 3.45 (s, 3H, 7′-OCH3), 3.72 (s, 3H, 6′-OCH₃), 5.93 (s, 1H, H8′), 6.72 (s, 1H, H5′), 6.93 (d, J=8.3 Hz, 1H, H7), 7.13 (d, J=2.1 Hz, 1H, H4), 7.25 (dd, J=8.3, 2.2 Hz, 1H, H6), 10.65 (s, 1H, H1). ¹³C NMR (DMSO-d₆, 100 MHz): δ ppm 22.5 (—CCH3), 36.6 (C4′), 45.0 (C3′), 55.9 (7′-OCH₃), 56.1 (6′-OCH3), 66.2 (C3/C1′), 109.0 (C8′), 111.9 (C7), 112.7 (C5′), 124.7 (C4), 126.0 (C3a), 126.2 (C8′a), 128.6 (C6), 129.3 (C4′a), 139.2 (C5), 140.6 (C7a), 147.7 (C7′), 148.7 (C6′), 179.3 (C2), FTMS+cESI: m/z 359.29 [M+1]⁺.

Biological Evaluation

Identification of Bioactive DSIIQs

Parasite Strains and Culture Conditions

Plasmodium falciparum 3D7 (chloroquine-sensitive) and Dd2 (multidrug resistant) strains were obtained from the Biodefense and Emerging Infections (BEI) Research Resources (Manassas, Va.) and maintained using a modified Trager and Jensen method (Trager & Jensen, 1976). Briefly, parasites were grown in fresh O⁺ human red blood cells at 3% (v/v) haematocrit in complete RPMI 1640 medium containing glutamax and NaHCO₃, and supplemented with 25 mM HEPES, 0.5% Albumax II, 1× hypoxanthine and 20 μg/mL gentamicin. Parasite cultures were incubated at 37° C. in a humidified atmosphere with 5% CO₂. Spent culture media were changed daily and the parasitemia were maintained at <10% by regular partial replacement of the culture with equivalent amounts of fresh un-infected RBCs. Giemsa-stained thin blood smears were examined microscopically under oil immersion to quantify parasitemia and observe parasite morphology. When needed, parasites were synchronized at the ring stage by sorbitol (5%) treatment (Radfar et al., 2009) and further cultivated through one complete cycle (48 h) prior to drug activity studies.

Compound Screening and Hit Confirmation Analysis

Compound screening for antiplasmodial activities was carried out in 96-well microtitration plates (Thermo Fisher Scientific) using the SYBR green I based fluorescence method as describe by (Smilkstein et al., 2004). By principle, the dye intercalates between double stranded DNA bases producing over 1000-folds increase in fluorescence emission when appropriately excited. Given that erythrocytes are enucleated and lack DNA, fluorescence produced is proportional to parasite density and DNA content. Compounds in dimethyl sulfoxide (DMSO) were diluted in RPMI 1640 and co-cultured with parasites (1% parasitemia and 1.5% hematocrit) in 96-well plates. The final drug concentrations for primary screening were 10 μM and 10-0.078 μM for the dose-response-based hit confirmation analyses and the final DMSO concentration was 0.1% in each culture well. Artemisinin and chloroquine (Sigma-Aldrich) at 1 μM were used as positive drug controls, while the solvent treated culture (0.1% DMSO) was used as negative drug control. The plates were incubated at 37° C. in a humidified atmosphere with 5% CO₂ for 72 h. Thereafter, parasite growth was assessed by a 1 in 2 dilution of SYBR green lysis buffer and treated parasite cultures. Briefly, 80 μL of parasitized erythrocytes were transferred to dark plates and 40 μL of SYBR green lysis buffer added. Plates were incubated in the dark for 30 min and fluorescence measured using a Fluoroskan Ascent multi-well plate reader with excitation and emission wavelengths at 485 and 538 nm, respectively. Mean half-maximal effective concentrations (EC₅₀) were derived by plotting percent growth inhibition against log drug concentration and fitting the response data to a variable slope sigmoidal curve-fit function using GraphPad Prism v5.0. EC50 values represent means±standard error from 2 independent assays. The values were normalized with values of both negative and positive controls and the Z factor was computed. Primary hits were defined as compounds inhibiting parasite growth by at least 50% when compared to the DMSO solvent control and active compounds were defined as compounds exhibiting complete dose-response curves within the tested range, with EC50 values less than 2 μM.

Cytotoxicity Testing of Bioactive DSIIQs

Viability of the cells was assessed by MTT (3-(4,5-dimethylthiazol-2-yl)-2,5 Diphenyltetrazolium Bromide)-based cell proliferation assay (Vybrant MTT Cell Proliferation Assay Kit V-1315) according to the manufacturer's instructions. By principle, the soluble MTT is reduced to an insoluble colored formazan product by mitochondrial succinate dehydrogenase in viable cells. Normal African Green Monkey Kidney Epithelial (Vero) and the Human Liposarcoma (SW872) cells were maintained in MEM supplemented with 10% FBS, 1% Glutamine and 1% penicillin-streptomycin. Confluent cells were trypsinized and seeded at a density of 2500 cells/well (total volume 90 μL) in 96-well plates and incubated for 24 h prior to drug treatment. Two-fold serial dilutions of the compounds (100-0.0488 μM) were added to the plates and incubated under humidified conditions at 37° C. for 48 h. Absorbance of the formed formazan product was measured at 550 nm wavelength using a SUNRISE microtiter plate reader.

Dose-response curves were plotted using GraphPad Prism v.5 and CC₅₀ values obtained. Selectivity indices (SI=CC₅₀/EC₅₀) were calculated as an indication of toxicity relative to the observed antiplasmodial activity.

Antiplasmodial Modes of Action of Test Compounds

Activity Against Different Developmental Stages of P. falciparum Parasites

The inhibitory effects of active compounds or reference drugs on parasite development and schizont rupture were determined by quantitative light microscopy as previously described (Lee et al., 2015). Briefly, synchronized cultures (1.5% hematocrit and 3% parasitemia) at different time-points (early rings, mid trophozoites and schizonts) were treated with either test compound or controls at 10 μM final concentration for 24 h under regular culture conditions. Following treatment, Giemsa-stained thin smears were prepared and parasites were counted in 1,000 erythrocytes per treatment. Stage proportions in test wells relative to solvent control wells were calculated and used to assess the in vitro effects of each compound on trophozoites and schizont development as well as merozoite egress and invasion.

Effect of Compounds on Hemoglobin Degradation

To investigate drug effects on hemoglobin degradation, early ring and mid-trophozoite stage parasites (1.5% hematocrit, 5% parasitemia) were co-cultured with the test compounds or experimental controls (0.1% DMSO, chloroquine, artemisinin NITD609 and E64) at a final concentration of 10 μM. The cultures were incubated at 37° C. and 5% CO₂ for 24 h. Following incubation, inhibitors were removed by centrifugation at 1800 rpm for 5 min. The pellets were washed in an equal volume of 1×PBS and re-suspended in same volume of 0.1% saponin (in 1×PBS) for 3 min. Isolated parasites were pelleted at 2500 rpm for 5 min and washed twice in an equal volume of 1×PBS. The resulting parasite pellets were permeabilized by treatment in 20 μL of 1% Triton X-100 (in 1×PBS) for 5 min. Intraparasitic hemoglobin content was measured at 550 nm using a Nanodrop spectrophotometer, and folds increase in intra-parasite hemoglobin content was calculated relative to the E64-treated controls.

Effect of Compounds on Vacuolar Lipid Dynamics

The effect of the compounds on intracellular lipid dynamics was assessed by measurement of intracellular lipid content using Oil Red O. Briefly, early rings or late trophozoite stage parasites (30 hpi) were treated with the compounds or controls for 24 h under regular culture conditions. Following treatment, inhibitors were removed by centrifugation at 2500 rpm for 5 min and pellets were washed in equal volumes of 1×PBS. The parasites were fixed with 4% paraformaldehyde for 30 min at room temperature and pelleted at 2500. Three volumes of Oil Red O (ORO) stock solution (5 mg/mL in 100% isopropanol) were diluted in two volumes of distilled water and filtered prior to use. About 100 μL of the ORO were added to parasite pellets in the tube and incubated for 30 minutes. Parasites were pelleted and washed twice in distilled water. Thereafter, parasites were deposited on pre-cleaned coverslips and air dried. Dried coverslips were mounted in DAPI antifade reagent onto cleaned slides, air dried and viewed under a 100× oil immersion lens and images acquired using a Leica DM1000 fluorescence microscope suite (LAS version 4.5).

Drug-Combination Studies

In view of the overlapping modes of action between the hybrids and some known antimalarial drugs in terms of targeted cellular processes, we investigated the interaction between these compounds at variable combination ratios. Malaria parasites were continuously cultured and synchronized as earlier described prior to the assay.

Drug interaction studies were performed as described by (Arrey Tarkang et al., 2014).

Briefly, two-fold drug dilutions were prepared using a variable potency ratio drug combination approach, starting at 5EC₅₀A: 0EC₅₀B to 0EC₅₀A: 5EC₅₀B in serum-free medium, where A and B represent the different partner molecules. The ring-stage parasitized erythrocytes (˜10 hpi) were diluted in complete medium to 1% parasitemia and 1.5% hematocrit and 90 μL was added in duplicate to 10 μL of the drug dilution in a 96-well plate.

The test was run for 72 h and terminated when the untreated parasites were at the early trophozoite stage of the second cycle. Parasite viability was assessed by the SYBR-Green I fluorescence-based assay as earlier described. Dose-response curves and EC50 values of each combination and drug alone were obtained using GraphPad Prism v.5.0 and Microsoft Excel was used to calculate mean EC50 values and the standard error of the mean.

The obtained EC50 values were used to calculate 50% fractional inhibitory concentrations (FIC50) and the combination indices (CI) were computed from the obtained FICs.

Where

$${FIC}_{50} = \frac{{EC}_{50} of drug in combination}{{EC}_{50} of drug alone} \\ CI = \sum FIC$$

Preliminary Hit Expansion and Optimization Studies

To generate more potent compounds needed for downstream lead discovery analyses (DMPK, in vivo efficacy and safety, target identification), structural derivatives of the selected hit compounds were synthesized targeting either the C5 and C6 positions of the oxindole fragment or N2 and C3 positions of THIQ fragment.

Compound activity was assessed by dose-response analyses and secondary hits with >5-fold improvement of antiplasmodial activities were selected for further lead discovery studies.

In Silico ADMET Analysis of Bioactive DSIIQs

As preliminary assessment of compound pharmacokinetics, drug likeness and lead likeness, hits selected on the basis of their promising antiplasmodial activities and negligible cytotoxic profiles were subjected to virtual screening using the online tools SwissADME (http://www.swissadme.ch/index.php) and PKCSM (http://biosig.unimelb.edu.au/pkcsm/prediction_single/), and using the oral drug chloroquine and phase 3 clinical compound NITD609 as reference compounds. Pharmacokinetic properties were studied using the boiled egg model, allowing for intuitive evaluation of passive gastrointestinal absorption and brain penetration. A support vector model was also used to assess compound suitability as a P-glycoprotein substrate (propensity for transmembrane uptake or efflux) or inhibition of various cytochrome 450 isoforms (propensity for metabolic elimination). Drug elimination was assessed based on predicted clearance rates and ability to serve as substrate for renal OCT2. Drug likeness was predicted on the basis of limited violations of Lipinski's rule of five (<2 violations) and based on a bioavailability score (probability of F>10% in rats). Compound lead likeness was determined based on accessibility to medicinal chemistry synthesis (from a score of 1 (very easy) to 10 (very difficult)). Furthermore, predictions were made on the mutagenic potential, cardiotoxicity, hepatotoxicity, rat acute or chronic toxicity and skin sensitization by using different models in the PKCSM ADMET prediction package.

In Vivo Mouse Efficacy of Selected Compounds

Mice Maintenance and Infection

Eight-week old male Swiss albino mice weighing approximately 23±3 g were housed in cages and maintained in a well-ventilated room under standard environmental conditions of temperature at 22-24° C., under a 12 h dark-light cycle, with food and water provided ad libitum.

The animals were allowed one week acclimation before commencement of the study. Three (3) mice were infected with stocked blood containing Plasmodium chabaudi parasites and used as donors. In brief, frozen blood containing the sulfadoxine/pyrimethamine resistant P. c. chabaudi (AS(50S/P) parasites was thawed and about 200 μL parasites were injected intra-peritoneally (i.p) into each of the three mice and parasitemia monitored through blood smear until a 20% threshold was reached. These infected animals then served as donors to the experimental animals. Ethical approval for the study was obtained from the University of Douala Institutional Review Board (N^{o}IEC-UD/1146/09/2017/A).

Chemosuppressive Activity of Selected Compounds

In vivo efficacies were conducted following a modification of the Peter's four-day suppressive test as previously described (Lee et al., 2014, Tarkang et al., 2014). Briefly, following parasite passage in donor mice, the mice were euthanized using diethyl ether and P. c. chabaudi infected blood was obtained by cardiac puncture and placed into heparinized tubes. The blood was then diluted with phosphate buffered saline (PBS) and immediately used to infect the experimental mice. Mice were randomly divided into seven (7) groups of 3 animals and each mouse was injected with 1×10⁷ infected erythrocytes intraperitoneally (Day 0). The mice were kept for 24 hours to establish infection and Giemsa thin blood smears were prepared to quantify parasites (Day 1). Thereafter, groups 1, 2, 3, 4 and 5 were treated with compounds dissolved in 1×PBS, intraperitoneally at dosages of 30, 10, 3, 0.3 and 0.03 mg/kg body weight, respectively, while groups 6 and 7 were treated with vehicle or chloroquine at 10 mg/kg, respectively for three consecutive days (Day 1-Day 3). Twenty-four (24) and ninety-six (96) hours post-infection, thin blood smears were prepared from each animal with blood obtained from the tail vein, fixed in methanol and stained with 10% Giemsa.

Parasitemia was determined by light microscopy using a 100× objective lens and the following equation:

$$\% parasitemia = \frac{No . of parasitized RBC}{Total no . of RBC counted} \times 100$$

Average percentage chemosuppression was calculated as

$$100 ( \frac{A - B}{A} )$$

Where A is mean parasitemia in negative control and B is mean parasitemia in test group

Compound ED₅₀ (dose resulting in a 50% reduction in parasitemia) and ED₉₀ (dose resulting in a 90% reduction in parasitemia) were further calculated by using the online tool, ED₅₀ Calculator (https://www.aatbio.com/tools/ed50-calculator).

Chemo-Curative Activity of Selected Compounds

The curative potential of each selected compound was evaluated using a modification of the Rane's test (Ryley & Peters, 1970). A standard inoculum of 10⁷ infected erythrocytes was injected per mouse intraperitoneally. A group of seven mice were left uninfected for the duration of the study in order to monitor for any behavioral changes due to infection with the parasite or treatment with the experimental compounds. Seventy-two hours later, the mice were randomly distributed into respective groups and dosed accordingly once daily for 5 days and intraperitoneally. The experimental compounds were dosed at their calculated ED₅₀ and ED₉₀ values, whereas the treated controls were dosed with chloroquine at 10 mg/kg body weight. A Giemsa-stained thin blood smear was prepared from the tail blood of each infected mouse on specific days up to day 29 post-infection to monitor the effect of treatment on blood parasitemia. The survival time for each group of mice was determined by calculating the average survival time (days) of the mice over the 30 days study duration (Days 0-29). Rectal temperatures and body weights were also recorded to detect any adverse effects of the treatment.

### REFERENCES

Agarwal D, Gupta R D, Awasthi S K (2017) Are antimalarial hybrid molecules a close reality or a distant dream? Antimicrob. Agents Chemother. 61(5): e00249-17.
Andrews E S, John A R O (2018) Tackling resistance: emerging antimalarials and new parasite targets in the era of elimination. F1000Research, 7(F1000 Faculty Rev): 1170 Last updated: 1 Aug. 2018.
Ashton T D, Devine S M, Möhrle J J, Laleu B, Burrows J N, Charman S A, Creek D J, Sleebs B E. The development process for discovery and clinical advancement of modern antimalarials. J. Med. Chem. 2019, 62, 10526-10562.
Bennett T N, Paguio M, Gligorijevic B, Seudieu C, Kosar A D, Davidson E, Roepe P D (2004) Antimicrob. Agents Chemother. 48, 1807e1810.
Biamonte M A, Wanner J, Le Roch K G (2013) Recent advances in malaria drug discovery. Bioorg. Med. Chem. Lett. 23: 2829-2843.
Bringmann G, Bischof S K, Müller S, Gulder T, Winter C, Stich A, Moll H, Kaiser M, Brun R, Dreher J, Baumann K (2010) QSAR guided synthesis of simplified antiplasmodial analogues of naphthylisoquinoline alkaloids. Eur. J. Med. Chem. 45: 5370-5383.
Fayez S, Feineis D, Assi L A, Marcel Kaiser M, Brun R, Awale S, Bringmann G (2018) Ancistrobrevines E-J and related naphthylisoquinoline alkaloids from the West African liana Ancistrocladus abbreviatus with inhibitory activities against Plasmodium falciparum and PANC-1 human pancreatic cancer cells. Fitoterapia 131: 245-259.
Francois G, Chimanuka B, Timperman G, Holenz J, Plaizier-Vercammen J, Assi L A, Bringmann G (1999) Differential sensitivity of erythrocytic stages of the rodent malaria parasite Plasmodium chabaudi to dioncophylline B, a highly active naphthylisoquinoline alkaloid. Parasitol Res. 85: 935-941.
Francois G, Timperman G, Eling W, Assi L A, Holenz J, Bringmann G (1997a) Naphthylisoquinoline alkaloids against malaria: Evaluation of the curative potentials of dioncophylline C and dioncopeltine A against Plasmodium berghei in vivo. Antimicrob. Agents Chemother. 41: 2533-2539.
Ibrahim S R M & Mohamed G A (2015) Naphthylisoquinoline alkaloids potential drug leads. Fitoterapia 106: 194-225.
Kweyamba P A, Zofou D, Efange N, Assob J-C N, Kitau J, Nyindo M (2019) In vitro and in vivo studies on anti-malarial activity of Commiphora Africana and Dichrostachys cinerea used by the Maasai in Arusha region, Tanzania. Malar J (2019) 18:119.
Li J, Seupel R, Feineis D, Mudogo V, Kaiser M, Brun R, Brunnert D, Chatterjee M, Seo E, Efferth T, Bringmann G (2017) Dioncophyllines C2, D2, and F and related naphthylisoquinoline alkaloids from the Congolese liana Ancistrocladus ileboensis with potent activities against Plasmodium falciparum and against multiple myeloma and leukemia cell lines. J. Nat. Prod. 80: 443-458.
Lobe M M M, Efange S M N (2020) 3′,4′-Dihydro-2′H-spiro[indolin-3:1′-isoquinolin]-2-ones as potential anticancer agents: synthesis and preliminary screening. R. Soc. open sci. 7: 191316. http://dx.doi.org/10.1098/rsos.191316.
Ngo Hanna J, Ntie-Kang F, Kaiser M, Brun R & Efange S M N (2014) 1-Aryl-1,2,3,4-tetrahydroisoquinolines as potential antimalarials: synthesis, in vitro antiplasmodial activity and in silico pharmacokinetics evaluation. RSC Adv. 4: 22856-22865.
Pedrosa M O, da Cruz R M D, Viana J O, de Moura R O et al. (2017) Hybrid compounds as direct multitarget ligands: a review. Curr. Top. Med. Chem. 17: 1044-1079.
Roberts B F, Zheng Y, Cleaveleand J, Lee S, Lee E, Ayong L, Yuan Y, Chakrabarti D (2017) 4-Nitro styrylquinoline is an antimalarial inhibiting multiple stages of Plasmodium falciparum asexual life cycle. International Journal for Parasitology: Drugs and Drug Resistance 7 (2017) 120e129.
Rottmann M, McNamara C, Yeung BKS, Lee MCS et al. (2010) Spiroindolones, a potent compound class for the treatment of malaria. Science 329 (3 Sep. 2010) 1175.
Smith P W, Diagana T T, Yeung B K S (2014) Progressing the global antimalarial portfolio: finding drugs which target multiple Plasmodium life stages. Parasitology 141:66-76.
Tarkang P A, Frabzoi K D, Lee S, Lee E et al. (2014) In vitro Antiplasmodial activities and synergistic combinations of differential solvent extracts of the polyherbal product, Nefang. Biomed Res. Intl. Vol. 2014, Article ID 835013. 10 pageshttp://dx.doi.org/10.1155/2014/835013.
Tibon N S, Ng C H, Cheong S L (2020) Current progress in antimalarial pharmacotherapy and multi-target drug discovery. Eur. J. Med. Chem. 188: 111983.
Tse E G, Korsik M, Todd M H (2019) The past, present and future of anti-malarial medicines. Malar J. (2019) 18:93.
Turner H (2016). Spiroindolone NITD609 is a novel antimalarial drug that targets the P-type ATPase Pf ATP4. Future Med. Chem. 8(2) pp. 227-238.
Yeung B K S, Zou B, Rottmann M, Lakshminarayana S B et al. (2010) Spirotetrahydro-β-carbolines (spiroindolones): A new class of potent and orally efficacious compounds for the treatment of malaria. J. Med. Chem. 53: 5155-5164.

1. A method of treating malaria in a patient or subject in need comprising administering to said patient or subject a therapeutically effective amount of at least one compound according to the chemical structure:
I: 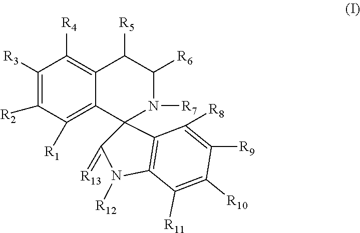
wherein R₁ is H, OH, C₁-C₆ hydroxyalkyl, halo (F, Cl, Br, I), C₁-C₆ alkoxy (often C₁-C₃ alkoxy, more often OMe), (CH₂)ₙCOOH, (CH₂)ₙC(O)C₀-C₆ alkyl, (CH₂)ₙC(O)OC₁-C₆ alkyl, (CH₂)ₙOC(O)C₀-C₆ alkyl or O(CH₂)ₙaryl, aryl, haloaryl, alkoxyaryl (more often phenyl or naphthyl).
R₂ and R₃ are each independently H, OH, C₁-C₆ hydroxyalkyl, halo (F, Cl, Br, I), C₁-C₆ alkoxy (often C₁-C₃ alkoxy, more often OMe), (CH₂)ₙCOOH, (CH₂)ₙC(O)C₀-C₆ alkyl, (CH₂)ₙC(O)OC₁-C₆ alkyl, (CH₂)ₙOC(O)C₀-C₆ alkyl, O—(CH₂)ₙaryl, aryl, haloaryl, alkoxyaryl (more often phenyl or naphthyl), heteroaryl (more often thienyl, furyl, pyrrolyl, pyridyl) or R₂ and R₃ together form a 5- or 6-membered cycloalkyl or heterocyclic group containing 1, 2 or 3 heteroatoms (O, S, or N), preferably, the heterocyclic group formed is a dioxolanyl (3,4-methylenedioxy), dioxanyl (3,4-ethylenedioxy), dithiolanyl, tetrahydrofuranyl, tetrahydrothiophenyl, pyrrolidinyl, imidazolyl, imidazolinyl, imidazolidinyl, tetrahydropyranyl, thienyl, piperidinyl or piperazinyl;
R₄ is H, OH, halo (F, Cl, Br, I), C₁-C₆ alkoxy (often C₁-C₃ alkoxy, more often OMe), (CH₂)ₙCOOH, (CH₂)ₙC(O)C₀-C₆ alkyl, (CH₂)ₙC(O)OC₁-C₆ alkyl, (CH₂)ₙOC(O)C₀-C₆ alkyl, O(CH₂)ₙaryl, (CH₂)ₙAryl (often, phenyl or naphthyl, more often phenyl), (CH₂)ₙHeteroaryl; aryl, alkoxyaryl, haloaryl (more often phenyl or naphthyl), heteroaryl (more often furyl, thienyl, pyrrolyl, pyridyl);
R₅ is H, alkyl (preferably C₁-C₆ alkyl), C₁-C₆ alkoxy, (CH₂)ₙAryl (often, phenyl or naphthyl, more often phenyl), (CH₂)ₙHeteroaryl, aryl (preferably, phenyl, substituted phenyl); heteroaryl (preferably, pyridyl, thienyl, furyl, pyrrolyl);
R₆ is H, alkyl (preferably C₁-C₆ alkyl), trifluoromethylalkyl (preferably C₁-C₅ alkyl), C₁-C₆ alkoxy, (CH₂)ₙAryl (often, phenyl or naphthyl, more often phenyl), (CH₂)ₙHeteroaryl, carboxyl, carbomethoxy, carboxamido, cyano;
R₇ is H, alkyl (preferably C₁-C₆ alkyl), (CH₂)ₙAryl (often, phenyl or naphthyl, more often phenyl), (CH₂)ₙC(O)C₀-C₆ alkyl, —(CH₂)ₙR^{N1}N—C(O)—NR^{N2}R^{N3}, (CH₂)ₙ—S(O)₂Aryl, —OC(O)NR^{N1}R^{N2},
R₈ is H, OH, Halo, Nitro, C₁-C₆ hydroxyalkyl, (CH₂)ₙNR^{N1}R^{N2}, —(CH₂)ₙ—NR^{N1}—(CH₂)ₙ-Aryl (often, phenyl or naphthyl, more often phenyl), —NR^{N1}SO₂Aryl (often, phenyl or naphthyl, more often phenyl), (CH₂)ₙC₃-C₈cycloalkyl-NR^{N1}R^{N2}, C₁-C₆ alkoxy, O(CH₂)ₙaryl, (CH₂)ₙAryl (often, phenyl or naphthyl, more often phenyl), (CH₂)ₙHeteroaryl, C₁-C₆ alkyl, C₂-C₆ vinyl, C₂-C₆ alkynyl, —SO₂NR^{N1}R^{N2}, —OC(O)NR^{N1}R^{N2}, CONR^{N1}R^{N2}, CH₂NR^{N1}R^{N2}
R₉, R₁₀ and R₁₁ are each independently H, OH, Halo, Nitro, C₁-C₆ hydroxyalkyl, (CH₂)ₙNR^{N1}R^{N2}, —(CH₂)ₙ—NR^{N1}—(CH₂)ₙ-Aryl (often, phenyl or naphthyl, more often phenyl), —NR^{N1}SO₂Aryl (often, phenyl or naphthyl, more often phenyl), (CH₂)ₙC₃-C₈cycloalkyl-NR^{N1}R^{N2}, C₁-C₆ alkoxy, O(CH₂)ₙaryl, (CH₂)ₙAryl (often, phenyl or naphthyl, more often phenyl), (CH₂)ₙHeteroaryl, C₁-C₆ alkyl, C₂-C₆ vinyl, C₂-C₆ alkynyl, —SO₂NR^{N1}R^{N2}, —OC(O)NR^{N1}R^{N2}, (CH₂)ₙC(O)OC₀-C₆ alkyl or (CH₂)ₙOC(O)C₀-C₆ alkyl, CONR^{N1}R^{N2}, CH₂NR^{N1}R^{N2};
R₁₂ is H, OH, alkyl (C₁-C₆), hydroxyalkyl (preferably C₁-C₆ hydroxyalkyl), an optionally substituted (CH₂)ₙAryl (often, phenyl, benzyl or naphthyl, more often benzyl or naphthyl, the Aryl group being optionally substituted with one or two Halo groups, preferably F, Cl or Br, a nitro, CN or a C₁-C₆, preferably a C₁-C₃ alkyl group, preferably R₁₂ is an optionally substituted benzyl group or naphthyl group), (CH₂)ₙC₃-C₈cycloalkyl, (CH₂)ₙC(O)NR^{N1}Aryl or (CH₂)ₙ—C(O)C₀-C₆ alkyl, aryl (more often phenyl or naphthyl), heteroaryl (more often furyl, thienyl, pyrrolidyl or pyridyl);
R₁₃ is O or S;
R^{N1}, R^{N2} and R^{N3} are each independently H or a C₁-C₆ alkyl group which is optionally substituted with one or two hydroxyl groups and up to three halo groups (preferably F);
n is 0-12, preferably 0-6, often 0, 1, 2 or 3, or
a pharmaceutically acceptable salt, stereoisomer, solvate, polymorph or mixture thereof.

2. The method according to claim 1 wherein R₁₂ is a phenyl group, a benzyl group or a naphthyl group, each of which is optionally substituted with a C₁-C₆ alkyl group, a nitro group, a cyano group or one or two halo groups (preferably F, Cl or Br).

3. The method according to claim 1 wherein said compound is a compound as set forth in FIG. 1 hereof or a pharmaceutically acceptable salt thereof.

4. The method according to claim 1 wherein the compound is selected from the group consisting of compounds 4a-c,d,f-h,j-l; 5b,d,e-l,o; 6a-c; 7a-d,h,i; 8a-c,h-m, and 9a-c,h,i as set forth in FIG. 1, or a pharmaceutically acceptable salt thereof.

5. The method according to claim 1 wherein the compound is a compound 10, 11 or 12 of FIG. 1, or a pharmaceutically acceptable salt thereof.

6. The method according to claim 1, wherein R₁, R₂ and R₃ are each independently H, halo or methoxy and R₄ is hydrogen, methyl or phenyl, wherein said phenyl group is optionally substituted with 1 or 2 halo groups, a nitro group, a CN group or a C₁-C₆ alkyl group.

7. The method according to claim 1, where the compound is as set forth in FIG. 1 hereof.

8. The method according to any of claims 1-7 wherein said compound is a mixture of two compounds.

9. The method according to any one of claims 1-8 wherein said malaria is caused by a parasite selected from the group consisting of P. falciparum, P. vivax, P. ovale, P. malariae and P. knowlesi.

10. The method according to any one of claims 1-9 wherein said malaria is caused by P. falciparum.

11. The method according to any one of claims 1-10 wherein at least one compound as set forth in FIG. 2 or a pharmaceutically acceptable salt hereof is co-administered with said compound according to chemical structure I.

12. The method according to any one of claims 1-11 wherein an additional drug co-administered with said compound, the drug combination being selected from the group consisting of artemether-lumefantrine, artesunate-amodiaquine, atovaquone-proguanil, quinine sulfate-doxycycline or a pharmaceutically acceptable salt thereof.

13. The method according to any of claims 1-12 wherein said treatment results in a cure of malaria.

14. A method of inhibiting or reducing the likelihood of malaria in a subject at risk for contracting malaria comprising administering to said subject a therapeutically effective amount of a compound according to the chemical structure I: 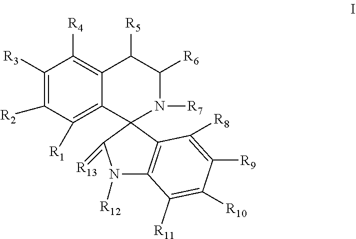
wherein R₁ is H, OH, C₁-C₆ hydroxyalkyl, halo (F, Cl, Br, I), C₁-C₆ alkoxy (often C₁-C₃ alkoxy, more often OMe), (CH₂)ₙCOOH, (CH₂)ₙC(O)C₀-C₆ alkyl, (CH₂)ₙC(O)OC₁-C₆ alkyl, (CH₂)ₙOC(O)C₀-C₆ alkyl or O(CH₂)ₙaryl, aryl, haloaryl, alkoxyaryl (more often phenyl or naphthyl).
R₂ and R₃ are each independently H, OH, C₁-C₆ hydroxyalkyl, halo (F, Cl, Br, I), C₁-C₆ alkoxy (often C₁-C₃ alkoxy, more often OMe), (CH₂)ₙCOOH, (CH₂)ₙC(O)C₀-C₆ alkyl, (CH₂)ₙC(O)OC₁-C₆ alkyl, (CH₂)ₙOC(O)C₀-C₆ alkyl, O—(CH₂)ₙaryl, aryl, haloaryl, alkoxyaryl (more often phenyl or naphthyl), heteroaryl (more often thienyl, furyl, pyrrolyl, pyridyl) or R₂ and R₃ together form a 5- or 6-membered cycloalkyl or heterocyclic group containing 1, 2 or 3 heteroatoms (O, S, or N), preferably, the heterocyclic group formed is a dioxolanyl (3,4-methylenedioxy), dioxanyl (3,4-ethylenedioxy), dithiolanyl, tetrahydrofuranyl, tetrahydrothiophenyl, pyrrolidinyl, imidazolyl, imidazolinyl, imidazolidinyl, tetrahydropyranyl, thienyl, piperidinyl or piperazinyl;
R₄ is H, OH, halo (F, Cl, Br, I), C₁-C₆ alkoxy (often C₁-C₃ alkoxy, more often OMe), (CH₂)ₙCOOH, (CH₂)ₙC(O)C₀-C₆ alkyl, (CH₂)ₙC(O)OC₁-C₆ alkyl, (CH₂)ₙOC(O)C₀-C₆ alkyl, O(CH₂)ₙaryl, (CH₂)ₙAryl (often, phenyl or naphthyl, more often phenyl), (CH₂)ₙHeteroaryl; aryl, alkoxyaryl, haloaryl (more often phenyl or naphthyl), heteroaryl (more often furyl, thienyl, pyrrolyl, pyridyl);
R₅ is H, alkyl (preferably C₁-C₆ alkyl), C₁-C₆ alkoxy, (CH₂)ₙAryl (often, phenyl or naphthyl, more often phenyl), (CH₂)ₙHeteroaryl, aryl (preferably, phenyl, substituted phenyl); heteroaryl (preferably, pyridyl, thienyl, furyl, pyrrolyl);
R₆ is H, alkyl (preferably C₁-C₆ alkyl), trifluoromethylalkyl (preferably C₁-C₅ alkyl), C₁-C₆ alkoxy, (CH₂)ₙAryl (often, phenyl or naphthyl, more often phenyl), (CH₂)ₙHeteroaryl, carboxyl, carbomethoxy, carboxamido, cyano;
R₇ is H, alkyl (preferably C₁-C₆ alkyl), (CH₂)ₙAryl (often, phenyl or naphthyl, more often phenyl), (CH₂)ₙC(O)C₀-C₆ alkyl, —(CH₂)ₙR^{N1}N—C(O)—NR^{N2}R^{N3}, (CH₂)ₙ—S(O)₂Aryl, —OC(O)NR^{N1}R^{N2},
R₈ is H, OH, Halo, Nitro, C₁-C₆ hydroxyalkyl, (CH₂)ₙNR^{N1}R^{N2}, —(CH₂)ₙ—NR^{N1}—(CH₂)ₙ-Aryl (often, phenyl or naphthyl, more often phenyl), —NR^{N1}SO₂Aryl (often, phenyl or naphthyl, more often phenyl), (CH₂)ₙC₃-C₈cycloalkyl-NR^{N1}R^{N2}, C₁-C₆ alkoxy, O(CH₂)ₙaryl, (CH₂)ₙAryl (often, phenyl or naphthyl, more often phenyl), (CH₂)ₙHeteroaryl, C₁-C₆ alkyl, C₂-C₆ vinyl, C₂-C₆ alkynyl, —SO₂NR^{N1}R^{N2}, —OC(O)NR^{N1}R^{N2}, CONR^{N1}R^{N2}, CH₂NR^{N1}R^{N2}
R₉, R₁₀ and R₁₁ are each independently H, OH, Halo, Nitro, C₁-C₆ hydroxyalkyl, (CH₂)ₙNR^{N1}R^{N2}, —(CH₂)ₙ—NR^{N1}—(CH₂)ₙ-Aryl (often, phenyl or naphthyl, more often phenyl), —NR^{N1}SO₂Aryl (often, phenyl or naphthyl, more often phenyl), (CH₂)ₙC₃-C₈cycloalkyl-NR^{N1}R^{N2}, C₁-C₆ alkoxy, O(CH₂)ₙaryl, (CH₂)ₙAryl (often, phenyl or naphthyl, more often phenyl), (CH₂)ₙHeteroaryl, C₁-C₆ alkyl, C₂-C₆ vinyl, C₂-C₆ alkynyl, —SO₂NR^{N1}R^{N2}, —OC(O)NR^{N1}R^{N2}, (CH₂)ₙC(O)OC₀-C₆ alkyl or (CH₂)ₙOC(O)C₀-C₆ alkyl, CONR^{N1}R^{N2}, CH₂NR^{N1}R^{N2};
R₁₂ is H, OH, alkyl (C₁-C₆), hydroxyalkyl (preferably C₁-C₆ hydroxyalkyl), an optionally substituted (CH₂)ₙAryl (often, phenyl, benzyl or naphthyl, more often benzyl or naphthyl, the Aryl group being optionally substituted with one or two Halo groups, preferably F, Cl or Br, a nitro, CN or a C₁-C₆, preferably a C₁-C₃ alkyl group, preferably R₁₂ is an optionally substituted benzyl group or naphthyl group), (CH₂)ₙC₃-C₈cycloalkyl, (CH₂)ₙC(O)NR^{N1}Aryl or (CH₂)ₙ—C(O)C₀-C₆ alkyl, aryl (more often phenyl or naphthyl), heteroaryl (more often furyl, thienyl, pyrrolidyl or pyridyl);
R₁₃ is O or S;
R^{N1}, R^{N2} and R^{N3} are each independently H or a C₁-C₆ alkyl group which is optionally substituted with one or two hydroxyl groups and up to three halo groups (preferably F);
n is 0-12, preferably 0-6, often 0, 1, 2 or 3, or
a pharmaceutically acceptable salt, stereoisomer, solvate, polymorph or mixture thereof.

15. The method according to claim 14 wherein R₁₂ is a phenyl group, a benzyl group or a naphthyl group, each of which is optionally substituted with a C₁-C₆ alkyl group, a nitro group, a cyano group or one or two halo groups (preferably F, Cl or Br).

16. The method according to claim 14 wherein said compound is a compound as set forth in FIG. 1 hereof or a pharmaceutically acceptable salt thereof.

17. The method according to claim 14 wherein the compound is selected from the group consisting of compounds 4a-c,d,f-h,j-l; 5b,d,e-l,o; 6a-c; 7a-d,h,i; 8a-c,h-m, and 9a-c,h,i as set forth in FIG. 1, or a pharmaceutically acceptable salt thereof.

18. The method according to claim 14 wherein the compound is a compound 10, 11 or 12 of FIG. 1, or a pharmaceutically acceptable salt thereof.

19. The method according to claim 14, wherein R₁, R₂ and R₃ are each independently H, halo or methoxy and R₄ is hydrogen, methyl or phenyl, wherein said phenyl group is optionally substituted with 1 or 2 halo groups, a nitro group, a CN group or a C₁-C₆ alkyl group.

20. The method according to claim 14, where the compound is as set forth in FIG. 1 hereof.

21. The method according to any of claims 14-20 wherein said compound is a mixture of two compounds.

22. The method according to any one of claims 14-21 wherein said malaria is caused by a parasite selected from the group consisting of P. falciparum, P. vivax, P. ovale, P. malariae and P. knowlesi.

23. The method according to any one of claims 14-22 wherein said malaria is caused by P. falciparum.

24. The method according to any one of claims 14-23 wherein said compound is co-administered with at least one compound as set forth in FIG. 2.

25. The method according to any one of claims 14-24 wherein said compound is co-administered with a drug selected from the group consisting of artemether-lumefantrine, artesunate-amodiaquine, atovaquone-proguanil and quinine sulfate-doxycycline.

26. A pharmaceutical composition comprising an effective amount of a compound according to the chemical structure I: 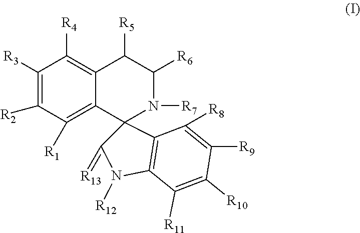
wherein R₁ is H, OH, C₁-C₆ hydroxyalkyl, halo (F, Cl, Br, I), C₁-C₆ alkoxy (often C₁-C₃ alkoxy, more often OMe), (CH₂)ₙCOOH, (CH₂)ₙC(O)C₀-C₆ alkyl, (CH₂)ₙC(O)OC₁-C₆ alkyl, (CH₂)ₙOC(O)C₀-C₆ alkyl or O(CH₂)ₙaryl, aryl, haloaryl, alkoxyaryl (more often phenyl or naphthyl).
R₂ and R₃ are each independently H, OH, C₁-C₆ hydroxyalkyl, halo (F, Cl, Br, I), C₁-C₆ alkoxy (often C₁-C₃ alkoxy, more often OMe), (CH₂)ₙCOOH, (CH₂)ₙC(O)C₀-C₆ alkyl, (CH₂)ₙC(O)OC₁-C₆ alkyl, (CH₂)ₙOC(O)C₀-C₆ alkyl, O—(CH₂)ₙaryl, aryl, haloaryl, alkoxyaryl (more often phenyl or naphthyl), heteroaryl (more often thienyl, furyl, pyrrolyl, pyridyl) or R₂ and R₃ together form a 5- or 6-membered cycloalkyl or heterocyclic group containing 1, 2 or 3 heteroatoms (O, S, or N), preferably, the heterocyclic group formed is a dioxolanyl (3,4-methylenedioxy), dioxanyl (3,4-ethylenedioxy), dithiolanyl, tetrahydrofuranyl, tetrahydrothiophenyl, pyrrolidinyl, imidazolyl, imidazolinyl, imidazolidinyl, tetrahydropyranyl, thienyl, piperidinyl or piperazinyl;
R₄ is H, OH, halo (F, Cl, Br, I), C₁-C₆ alkoxy (often C₁-C₃ alkoxy, more often OMe), (CH₂)ₙCOOH, (CH₂)ₙC(O)C₀-C₆ alkyl, (CH₂)ₙC(O)OC₁-C₆ alkyl, (CH₂)ₙOC(O)C₀-C₆ alkyl, O(CH₂)ₙaryl, (CH₂)ₙAryl (often, phenyl or naphthyl, more often phenyl), (CH₂)ₙHeteroaryl; aryl, alkoxyaryl, haloaryl (more often phenyl or naphthyl), heteroaryl (more often furyl, thienyl, pyrrolyl, pyridyl);
R₅ is H, alkyl (preferably C₁-C₆ alkyl), C₁-C₆ alkoxy, (CH₂)ₙAryl (often, phenyl or naphthyl, more often phenyl), (CH₂)ₙHeteroaryl, aryl (preferably, phenyl, substituted phenyl); heteroaryl (preferably, pyridyl, thienyl, furyl, pyrrolyl);
R₆ is H, alkyl (preferably C₁-C₆ alkyl), trifluoromethylalkyl (preferably C₁-C₅ alkyl), C₁-C₆ alkoxy, (CH₂)ₙAryl (often, phenyl or naphthyl, more often phenyl), (CH₂)ₙHeteroaryl, carboxyl, carbomethoxy, carboxamido, cyano;
R₇ is H, alkyl (preferably C₁-C₆ alkyl), (CH₂)ₙAryl (often, phenyl or naphthyl, more often phenyl), (CH₂)ₙC(O)C₀-C₆ alkyl, —(CH₂)ₙR^{N1}N—C(O)—NR^{N2}R^{N3}, (CH₂)ₙ—S(O)₂Aryl, —OC(O)NR^{N1}R^{N2},
R₈ is H, OH, Halo, Nitro, C₁-C₆ hydroxyalkyl, (CH₂)ₙNR^{N1}R^{N2}, —(CH₂)ₙ—NR^{N1}—(CH₂)ₙ-Aryl (often, phenyl or naphthyl, more often phenyl), —NR^{N1}SO₂Aryl (often, phenyl or naphthyl, more often phenyl), (CH₂)ₙC₃-C₈cycloalkyl-NR^{N1}R^{N2}, C₁-C₆ alkoxy, O(CH₂)ₙaryl, (CH₂)ₙAryl (often, phenyl or naphthyl, more often phenyl), (CH₂)ₙHeteroaryl, C₁-C₆ alkyl, C₂-C₆ vinyl, C₂-C₆ alkynyl, —SO₂NR^{N1}R^{N2}, —OC(O)NR^{N1}R^{N2}, CONR^{N1}R^{N2}, CH₂NR^{N1}R^{N2}
R₉, R₁₀ and R₁₁ are each independently H, OH, Halo, Nitro, C₁-C₆ hydroxyalkyl, (CH₂)ₙNR^{N1}R^{N2}, —(CH₂)ₙ—NR^{N1}—(CH₂)ₙ-Aryl (often, phenyl or naphthyl, more often phenyl), —NR^{N1}SO₂Aryl (often, phenyl or naphthyl, more often phenyl), (CH₂)ₙC₃-C₈cycloalkyl-NR^{N1}R^{N2}, C₁-C₆ alkoxy, O(CH₂)ₙaryl, (CH₂)ₙAryl (often, phenyl or naphthyl, more often phenyl), (CH₂)ₙHeteroaryl, C₁-C₆ alkyl, C₂-C₆ vinyl, C₂-C₆ alkynyl, —SO₂NR^{N1}R^{N2}, —OC(O)NR^{N1}R^{N2}, (CH₂)ₙC(O)OC₀-C₆ alkyl or (CH₂)ₙOC(O)C₀-C₆ alkyl, CONR^{N1}R^{N2}, CH₂NR^{N1}R^{N2};
R₁₂ is H, OH, alkyl (C₁-C₆), hydroxyalkyl (preferably C₁-C₆ hydroxyalkyl), an optionally substituted (CH₂)ₙAryl (often, phenyl, benzyl or naphthyl, more often benzyl or naphthyl, the Aryl group being optionally substituted with one or two Halo groups, preferably F, Cl or Br, a nitro, CN or a C₁-C₆, preferably a C₁-C₃ alkyl group, preferably R₁₂ is an optionally substituted benzyl group or naphthyl group), (CH₂)ₙC₃-C₈cycloalkyl, (CH₂)ₙC(O)NR^{N1}Aryl or (CH₂)ₙ—C(O)C₀-C₆ alkyl, aryl (more often phenyl or naphthyl), heteroaryl (more often furyl, thienyl, pyrrolidyl or pyridyl);
R₁₃ is O or S;
R^{N1}, R^{N2} and R^{N3} are each independently H or a C₁-C₆ alkyl group which is optionally substituted with one or two hydroxyl groups and up to three halo groups (preferably F);
n is 0-12, preferably 0-6, often 0, 1, 2 or 3, or
a pharmaceutically acceptable salt, stereoisomer, solvate or polymorph thereof, in combination with a pharmaceutically acceptable carrier, additive or excipient.

27. The composition according to claim 26 comprising at least one additional anti-malarial agent.

28. The composition according to claim 26 or 27 wherein R₁₂ is a phenyl group, a benzyl group or a naphthyl group, each of which is optionally substituted with a C₁-C₆ alkyl group, a nitro group, a cyano group or one or two halo groups (preferably F, Cl or Br).

29. The composition according to claim 26 or 27 wherein said compound is a compound as set forth in FIG. 1 hereof or a pharmaceutically acceptable salt thereof.

30. The composition according to claim 26 or 27 wherein the compound is selected from the group consisting of compounds 4a-c,d,f-h,j-l; 5b,d,e-l,o; 6a-c; 7a-d,h,i; 8a-c,h-m, and 9a-c,h,i as set forth in FIG. 1, or a pharmaceutically acceptable salt thereof.

31. The composition according to claim 26 or 27 wherein the compound is a compound 10, 11 or 12 of FIG. 1, or a pharmaceutically acceptable salt thereof.

32. The composition according to claim 26 or 27, wherein R₁, R₂ and R₃ are each independently H, halo or methoxy and R₄ is hydrogen, methyl or phenyl, wherein said phenyl group is optionally substituted with 1 or 2 halo groups, a nitro group, a CN group or a C₁-C₆ alkyl group.

33. The composition according to any of claims 26-32 wherein said compound is a mixture of two compounds.

34. The composition according to any one of claims 26-33 wherein at least one compound as set forth in FIG. 2 hereof is included in said pharmaceutical composition.

35. The composition according to any one of claims 26-33 wherein said compound is included in said composition in combination with a compound selected from the group consisting of artemether-lumefantrine, artesunate-amodiaquine, atovaquone-proguanil and quinine sulfate-doxycycline.

36. The composition according to any of claims 26-35 in oral dosage form.

37. The composition according to any of claims 26-35 in parenteral dosage form.

38. A compound as set forth in FIG. 17 hereof or a pharmaceutically acceptable salt thereof.

39. A compound according to chemical structure II: 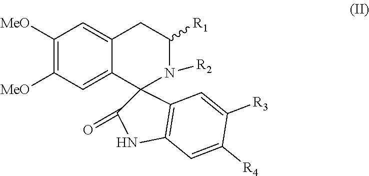
Wherein R₁ is hydrogen or methyl;
R₂ is hydrogen or methyl;
R₃ is halogen, preferably fluorine or chlorine; and
R₄ is halogen, preferably fluorine or chlorine or a nitrogen-containing heterocyle, preferably pyrrolidyl, pyrrolyl, piperidyl, morpholinyl or piperazinyl, or
a pharmaceutically acceptable salt thereof.

40. A pharmaceutical composition comprising an effective amount of a compound according to claim 38 in combination with a pharmaceutically effective amount of a carrier, additive or excipient.

41. A pharmaceutical composition comprising an effective amount of a compound according to claim 39 in combination with a pharmaceutically effective amount of a carrier, additive or excipient.

42. The composition according to claim 40 wherein said compound is a mixture of two compounds.

43. The composition according to claim 41 wherein said compound is a mixture of two compounds.

44. The composition according to any one of claims 40-43 wherein at least one compound as set forth in FIG. 2 hereof is included in said pharmaceutical composition.

45. The composition according to any one of claims 40-43 which includes a compound selected from the group consisting of artemether-lumefantrine, artesunate-amodiaquine, atovaquone-proguanil and quinine sulfate-doxycycline.

46. The composition according to any of claims 40-45 in oral dosage form.

47. The composition according to any of claims 40-45 in parenteral dosage form.

48. A method of inhibiting the growth or increase in population of a parasite selected from the group consisting of P. falciparum, P. vivax, P. ovale, P. malariae and P. knowlesi in a patient or subject in need comprising administering a composition according to any one of claims 26-37 to said patient or subject.

49. The method according to claim 48 wherein said parasite is P. falciparum.
